(19) United States
(12) Patent Application Publication
(10) Pub. No.: US 2019/0374158 A1
(43) Pub. Date: Dec. 12, 2019

(54) SYSTEM AND METHOD FOR MONITORING AND CONTROLLING A STATE OF A PATIENT DURING AND AFTER ADMINISTRATION OF ANESTHETIC COMPOUND

(71) Applicant: The General Hospital Corporation, Boston, MA (US)

(72) Inventors: Emery N. Brown, Brookline, MA (US); Patrick L. Purdon, Somerville, MA (US)

(21) Appl. No.: 16/293,325

(22) Filed: Mar. 5, 2019

Related U.S. Application Data
(63) Continuation of application No. 14/151,412, filed on Jan. 9, 2014, now abandoned.
Application No. 14/151,412 is a continuation-in-part of application No. PCT/US2013/064852, filed on Oct. 14, 2013.
(60) Provisional application No. 61/750,681, filed on Jan. 9, 2013.
(60) Provisional application No. 61/713,267, filed on Oct. 12, 2012.

(51) Int. Cl. A61B 5/00

(52) U.S. Cl. CPC A61B 5/4821; A61B 5/4839

(57) ABSTRACT

Systems and methods for monitoring and controlling anesthesia are provided. In some aspects, a method includes receiving data corresponding to EEG signals acquired from a patient and an indication of at least one characteristic of the patient and the at least one drug having anesthetic properties, and assembling, using the data received, one or more sets of EEG time-series. The method also includes selecting alpha frequency signals from the one or more sets of EEG time-series, and analyzing the alpha frequency signals to determine signatures particular to the at least one drug administered. The method further includes identifying at least one of a current state and a predicted future state of the patient induced by the at least one drug based on the signatures and the indication, and generating a report indicative of the at least one of the current state and the predicted future state of the patient.

### CROSS-REFERENCE TO RELATED APPLICATIONS

This application is a continuation of U.S. application Ser. No. 14/151,412 filed on Jan. 9, 2014 and entitled “SYSTEM AND METHOD FOR MONITORING AND CONTROLLING A STATE OF A PATIENT DURING AND AFTER ADMINISTRATION OF ANESTHETIC COMPOUND.” U.S. application Ser. No. 14/151,412 claims priority to PCT Application No. PCT/US2013/064852 filed Oct. 14, 2013 and entitled “SYSTEM AND METHOD FOR MONITORING AND CONTROLLING A STATE OF A PATIENT DURING AND AFTER ADMINISTRATION OF ANESTHETIC COMPOUND,” which further claims priority to U.S. Provisional Application Ser. No. 61/713,267 filed on Oct. 12, 2012 and entitled “SYSTEM AND METHOD FOR MONITORING AND CONTROLLING A STATE OF A PATIENT DURING AND AFTER ADMINISTRATION OF ANESTHETIC COMPOUND.” U.S. application Ser. No. 14/151,412 also claims priority to U.S. Provisional Application Ser. No. 61/750,681 filed on Jan. 9, 2013 and entitled “Intracranial EEG Signatures of Propofol General Anesthesia in Humans.”

### STATEMENT REGARDING FEDERALLY SPONSORED RESEARCH

This invention was made with government support under DP1 OD003646, DP2-OD006454, and K25-NS05758 awarded by the National Institutes of Health. The government has certain rights in the invention.

### BACKGROUND OF THE INVENTION

The present disclosure generally relates to systems and method for monitoring and controlling a state of a patient and, more particularly, to systems and methods for monitoring and controlling a state of a patient receiving a dose of anesthetic compound(s) or, more colloquially, receiving a dose of “anesthesia.”

Since 1846 and the first public uses of ether as a means to control pain during surgical procedures, anesthesia, analgesics, and other administered compounds to control pain and render patients unconscious have been a mainstay of medicine. However, while the use of the anesthetic and the number of compounds with anesthetic properties in clinical use have grown astronomically since the initial uses of ether, the scientific understanding of the operation of the body when under anesthesia is still developing. For example, a complete understanding of the effects of anesthesia on patients and operation of the patient's brain over the continuum of “levels” of anesthesia is still lacking. As such, anesthesiologists are trained to recognize the effects of anesthesia and extrapolate an estimate of the “level” of anesthetic influence on a given patient based on the identified effects of the administered anesthesia.

One common tool used by clinicians when monitoring patients receiving a dose of anesthesia is an electroencephalogram (EEG) system. EEG systems monitor electrophysiolgical signals of the brain. To provide the clinician with feedback, some EEG systems display a partial or amalgamized representation of the acquired signals as a waveform. However, as will be explained, many contemporary monitoring systems used during the administration of anesthesia provide feedback as a single dimensionless index that attempts to “quantify” the extremely-complex physiological responses of the patient receiving the dose of anesthesia and, thereby, convey the patient's depth of anesthesia. These EEG-based depth of anesthesia indices have been shown to poorly represent a patient's brain state, and moreover show substantial variability in underlying brain state and level of awareness at similar numerical values within and between patients. Recent advances in the neuroscience and neurophysiology of the anesthetic drugs show that different drugs act through different neural mechanism, producing different EEG signatures associated with different altered states of consciousness. These EEG signatures can vary across different anesthetic drugs. Analysis of the neural systems mechanisms for different anesthetic drugs can be found in Brown E N, Lydic R, & Schiff N D (2010) General anesthesia, sleep, and coma. New England Journal of Medicine 363(27):2638-2650 and in Brown E N, Purdon P L, & Van Dort C J (2011) General anesthesia and altered states of arousal: a systems neuroscience analysis. Annual Review of Neuroscience 34:601-628. The different EEG signatures associated with different altered states of consciousness induced by the commonly-used anesthetic drug propofol can be found in Purdon P L, Pierce E T, Mukamel E A, Prerau M J, Walsh J L, Wong K F K, Salazar-Gomez A F, Harrell P G, Sampson A, Cimenser A, Ching S, Kopell N, Tavares-Stoeckel C L, Habeeb K, Merhar R, Brown E N. Electroencephalogram signatures of loss and recovery of consciousness from propofol. Proceedings of the National Academy of Sciences, 2013 Mar. 19; 110(12):E1142-51. From the viewpoint of these recent advances, distinct anesthesia-related EEG signatures provide a more principled characterization of a patient's state under general anesthesia or sedation, and a more principled approach to controlling delivery of an anesthetic compound.

In practice, one common process that clinicians use is to monitor EEG display to identify indications of “burst suppression.” Burst suppression is an example of an EEG pattern that can be observed when the brain has severely reduced levels of neuronal activity, metabolic rate, and oxygen consumption. For example, burst suppression is commonly seen in profound states of general anesthesia. One example of a profound state of a patient under general anesthesia is medical coma. The burst suppression pattern often manifests as periods of bursts of electrical activity alternating with periods during which the EEG is isoelectric or suppressed. A variety of clinical scenarios require medical coma for purposes of brain protection, including treatment of uncontrolled seizures-status epilepticus- and brain protection following traumatic or hypoxic brain injury, anoxic brain injuries, hypothermia, and certain developmental disorders. Burst suppression represents a specific brain state resulting from such injuries, disorders, or medical interventions.

Traditional systems and methods that attempt to quantify burst suppression proceeds in two steps. First, characteristics of burst suppression are identified in the acquired data and the burst and suppression events are segregated or separated from EEG artifacts. Second, these systems and methods attempt to quantify the “level” of burst suppression.

For example, commercially available brain monitoring devices like those produced by G E, Covidien, and Masimo, use a so-called “suppression ratio” as part of an algorithm to identify and track the state of burst suppression. These algorithms are focused on segmenting the EEG into bursts and suppression periods and then quantify the identified information.

That is, several detection algorithms have been developed to accomplish the segregation or separation step. For example, many systems convert the EEG signal into a binary time series in which 1s correspond to suppression and 0s correspond to bursts. FIGS. 1 A and C, are EEG waveforms of respective 5-minute and a 1-minute segment illustrating burst suppression induced by the administration of the anesthetic propofol. FIGS. 1 B and D show the binary series associated with the raw signals of FIGS. 1A and C. Using this binary time series such as illustrated in FIGS. 1B and D, these commercially-available systems attempt to “quantify” the level of burst suppression. One common method of quantification is called a “burst suppression ratio” (BSR). The BSR quantifies the proportion of time, in a given time interval, that the EEG signal is designated as being suppressed by the segmentation step. The BSR is a fraction, ranging from 0, meaning no suppression to 1, meaning isoelectric or flat EEG.

Systems implementing BSR as a means for quantifying a state of a patient have been studied and positively correlated with a reduction in cerebral metabolic rate (CMR). During general anesthesia and during induced hypothermia, a state of complete electrical silence will be reflected by a BSR of one and the CMR decreases in a dose-dependent manner until it plateaus at a constant rate.

Although the importance of quantitatively analyzing burst suppression using, for example, a metric like BSR is broadly appreciated, there are key shortcomings with current approaches. For example, even though the 0s and 1s can be computed on intervals as short as 100 msec or even every milli-second, it is not unusual to use several seconds of these binary values to compute the BSR. This assumes that the brain state remains stable throughout the period during which the BSR is being computed. When the level of brain activity is changing rapidly, such as with induction of general anesthesia, hypothermia, or with rapidly evolving disease states, this assumption does not hold true. Instead, the computation of the level of burst suppression should match the resolution at which the binary events are recorded. Unfortunately, this reflects a practical quandary for the algorithm designer. Namely, the design cannot calculate a BSR without a determined time interval, but the true interval would be best selected with knowledge of the BSR to be calculated.

To further compound the difficulties of using such BSR algorithms clinically, different manufactures use different segmentation algorithms to convert the EEG into a binary time-series. Accordingly, different devices from different manufactures produce different BSR estimates. Comparing results across devices/manufacturer's is often challenging. As a further clinical challenge, for any of the situations in which burst suppression is tracked quantitatively, an important objective is to make formal statistical comparisons at different points in time. However, the statistical properties of the BSR estimated by averaging the binary events over several second intervals have not been described. As a consequence, there is no principled way to use the current BSR estimates in formal statistical analyses of burst suppression. That is, there is a lack of formal statistical analyses and prescribed protocols to implement formal statistical analyses to be able to state with a prescribed level of certainty that two or more brain states differ using current BSR protocols.

The shortcomings of these monitoring systems is compounded by the fact that they are often used as the information source on which clinicians make decisions. For example, referring to FIG. 2, a simplified schematic is illustrated showing that a “drug infusion” including a dose of anesthesia is delivered to a patient. Feedback from the patient is gathered by a monitoring system such as described above that attempts to identify and quantify burst suppression by providing an indication of “burst suppression level”. The “burst suppression level” is generally the amount of burst suppression perceived by the clinician looking at the monitor display. This “burst suppression level” then serves as the input to a clinician that serves as the control of a feedback loop by adjusting the drug infusion levels based on the indicated “burst suppression level.” This simplified example illustrates that errors or general inaccuracies in the “burst suppression level” indicated by the monitoring system and/or erroneous interpretations or assumptions by the clinician can exacerbate an already inexact process of controlling the drug infusion process. Such imprecision may be tolerable in some situations, but is highly unfavorable in others.

For example, in some clinical settings, it may be desirable to place a patient in a so-called “medical coma.” To do so, burst suppression is induced by manually tuning drug infusion to meet certain specifications. Control of these infusions requires the nursing staff to monitor, frequently by eye, the infusion pump and the EEG waveform, and to titrate the infusion rate of the anesthetic drug to achieve and maintain the desired EEG pattern. It is impractical for the nursing staff to provide a continuous assessment of the EEG waveform in relation to the rate of drug infusion in such a way to maintain tight control of the patient's desired brain state.

With these clinical challenges recognized, some have attempted to develop feedback and control systems the aid the clinician. For example, Bickford proposed an EEG-based, closed loop anesthetic delivery (CLAD) system more than 60 years ago. For example, a simplified schematic diagram of an early CLAD system is provided in FIG. 3. Bickford's original CLAD system of the 1950s used EEG content 300 in specific frequency bands as the control signal that indicated a current “depth of anesthesia” 302. The depth of anesthesia 302 was compared to a “target depth of anesthesia” 304, which determined with the drug infusion 306 should be increased or decreased. As such, a closed loop system was proposed to control the anesthetic delivered to the patient 308.

Later incarnations of the proposed CLAD systems used more sophisticated EEG analysis. For example, instead of simply relying on specific frequency bands as the control signal, systems were proposed that used metrics, such as the median frequency and the spectral edge, or the 50th and 95th quantiles of the power spectrogram, respectively. Studies observed a strong relationship between frequency content and its associated range and the corresponding depth of general anesthesia. Other possible control signals that were proposed included evoked potentials, or physiological responses, such as heart rate and blood pressure. Though commercial development of such systems did not begin in earnest until the 1980's, there have now been many clinical studies on the use of CLAD systems in anesthesiology practice and a system for sedation not using EEG is now commercially available.

Although CLAD systems have been around for many years and they are now used in anesthesiology practice outside of the United States, recent reports suggest that several problems with these systems have not been fully addressed. First, it has been recognized since 1937 that EEG patterns can serve as an indicator of brain state under general anesthesia. To date, sufficiently detailed quantitative analyses of the EEG waveform have not been performed to produce well-defined markers of how different anesthetic drugs or combinations of drugs alter the states of the patient and how such variations manifest in EEG waveforms and other physiological characteristics.

In an attempt to combat such problems, the so-called Bispectral Index (BIS) has been used an EEG-based marker to track brain state under general anesthesia and to provide a control signal for CLAD systems. BIS is derived by computing spectral and bispectral features of the EEG waveform. The features are input to a proprietary algorithm to derive an index between 0 and 100, in which 100 correspond to fully awake state with no drug effects and 0 corresponds to the most profound state of coma. As referenced above, BIS often serves as a common, single indicator clinicians rely upon to interpret the data acquired by a monitoring system. That is, clinicians simply rely upon the BIS indication to make clinical decisions.

As a control signal, BIS can inherently have only limited success, as the same BIS value can be produced by multiple distinct brain states. A patient under general anesthesia with isoflurane and oxygen, a patient sedated with dexmedetomidine, and a patient in stage III, or slow-wave, sleep can all have BIS values in the 40-to-60 range, which is the BIS interval in which surgery is conducted. Of these three patients, only the first is most likely in a state of “general anesthesia” and appropriate for conducting surgery. In this context, “general anesthesia” refers to unconsciousness, amnesia, analgesia, akinesia with maintenance of physiological stability. Similarly, patients anesthetized with ketamine alone or in combination with other anesthetic agents show high BIS values suggesting an awake or lightly sedated state, despite being in a state of general anesthesia. Although most reports nonetheless claim successful brain state control, such control has not been reliably demonstrated in individual subjects in a study or patients in real-time.

Second, using BIS to account for individual variability in response to anesthetic drugs and hence, in EEG patterns, under normal, surgical, and intensive care unit conditions is a challenge. Third, EEG processing by commercially-available monitors of anesthetic state is performed, not in real-time, but with a 20-to-30-second delay. Fourth, CLAD systems use ad-hoc algorithms instead of formal deterministic or stochastic control paradigms in their design. As a consequence, the reports in which CLAD systems have been implemented do not show reliable repeatable control results. Indeed, to give the appearance of successful control, the results of several subjects are often averaged in plots of CLAD performance. Finally, some have proposed the theoretical use of established control principles to design a CLAD system. However, such proposals have suggested the derivation of a wavelet-based index of anesthetic depth from the EEG, which fundamentally proposes a control signal that is analogous to BIS. Simply, until more is known about the neurophysiology of how EEG patterns relate to brain states under general anesthesia, developing generally applicable CLAD systems is a challenging problem. To this point, as described above, metrics such as BSR suffer from similar limitations and, thus, have not been suitable for developing generally applicable CLAD systems for at least the reasons discussed above.

Perhaps recognizing the complex nature of the EEG waveform and the shortcomings of BIS as a control system, Vijn and Sneyd designed CLAD systems for rats using a different metric, namely burst suppression ratio (BSR), as the control signal. BSR, is defined as the proportion of time per epoch that the EEG is suppressed below a predetermined voltage threshold. The BSR ranges from 0, meaning no suppression, to 1, meaning an isoelectric EEG. The objective of such investigation was to develop a model-free approach to CLAD-system design to determine if performance of new drugs in a CLAD system could provide useful information on drug design. They processed their error signal using a non-standard deterministic control strategy that was the product of a proportional and an integral term. Although the authors claim that their CLAD system maintained control of BSR for both propofol and etomidate, they reported BSR time courses averaged over groups of rats and not for individual animals. The Vijn and Sneyd CLAD system was recently implemented by Cotten et al. to test the efficacy of new etomidate-based anesthetics in controlling BSR in rats. These authors also reported only average time courses. Accordingly there seems to be a lack of studies on the use of CLAD systems to control burst suppression in human experiments or in the ICU to maintain a level of medical coma.

To further complicate matters, there are a great number of variables that can influence the effects, effectiveness, and, associated therewith, the “level” of anesthetic influence on a given patient. Thus, closed-loop control systems can fail if the drug infusion does not account for any of the plethora of variables. Some variables include physical attributes of the patient, such as age, state of general health, height, or weight, but also less obvious variables that are extrapolated, for example, based on prior experiences of the patient when under anesthesia. When these variables are compounded with the variables of a given control system or method and the variables presented by a particular anesthetic compound or, more so, combination of anesthetic compounds, the proper and effective administration of anesthesia to a given patient can appear to be an art, rather than a science.

In addition, whether controlled by a system, such as a CLAD system, or a more traditional clinician-specific control, emergence from general anesthesia is a slow passive process achieved simply by allowing the effects of the drug to wear off. Emergence from anesthesia is traditionally a passive process whereby anesthetic drugs are merely discontinued at the end of surgery, and no drugs are administered to actively reverse their effects on the brain and central nervous system. That is, the general anesthetic agents are merely discontinued at the end of surgery, leaving the anesthesiologist and surgeon to wait for the patient to recover consciousness. The timing of emergence can be unpredictable because many factors including the nature and duration of the surgery, and the age, physical condition and body habitus of the patient, can greatly affect the pharmacokinetics and pharmacodynamics of general anesthetics. Although the actions of many drugs used in anesthesiology can be pharmacologically reversed when no longer desired (e.g. muscle relaxants, opioids, benzodiazepines, and anticoagulants), this is not the case for general anesthetic induced loss of consciousness. While some basic ideas for actively reversing the effects of anesthesia have been considered, they do not translate well to traditional monitoring systems and control methods because these monitoring and control methods are generally unidirectional. For example, using burst-suppression based metrics for determining an increasing state of consciousness is counterintuitive, at best. Not surprisingly, then, control algorithms have not been developed to facilitate actively controlled recovery.

Considering the above, there continues to be a clear need for systems and methods to accurately monitor and quantify patient states and based thereon, provide systems and methods for controlling patient states during administration of anesthetic compounds.

### SUMMARY OF THE INVENTION

The present invention overcomes drawbacks of previous technologies by providing systems and methods that provide a number of advantages and capabilities not contemplated by, recognized in, or possible with traditional systems or known-methodologies related to the administration and control of anesthetic compounds.

In one embodiment, a system and method for monitoring and controlling the administration of at least one drug having anesthetic properties are provided. The system includes a plurality of sensors configured to acquire physiological data from the patient, and a user interface configured to receive an indication of at least one of a characteristic of the patient and the at least one drug having anesthetic properties. The system also includes at least one processor configured to review the physiological data from the plurality of sensors and the indication from the user interface. The processor is also configured to assemble the physiological data into sets of time-series data, analyze the sets of time-series data to determine signature profiles consistent with the administration of at least one drug having anesthetic properties, wherein the signature profiles are determined using signals from the time-series data in an alpha frequency range, and identify, using signature profiles, at least one of a current state and a predicted future state of the patient based on the indication. The processor is further configured to generate a report indicating at least one of the current state and the predicted future state of the patient induced by the drug.

In another embodiment, a method is provided for monitoring a patient experiencing an administration of at least one drug having anesthetic properties. The method includes arranging a plurality of sensors configured to acquire physiological data from a patient and reviewing the physiological data from the plurality of sensors and the indication from the user interface. The method also includes assembling the physiological data into sets of time-series data and analyzing the sets of time-series data to determine signature profiles consistent with the administration of at least one drug having anesthetic properties, wherein the signature profiles are determined using signals from the time-series data in an alpha frequency range. The method further includes identifying using signature profiles at least one of a current state and a predicted future state of the patient, based on the indication and generating a report including information regarding at least one of the current state and the predicted future state of the patient induced by the drug.

In yet another embodiment, a system is provided for monitoring and controlling a patient experiencing an administration of at least one drug having anesthetic properties. The system includes a plurality of sensors configured to acquire physiological data from the patient and a user interface configured to receive an indication of at least one of a characteristic of the patient and the at least one drug having anesthetic properties. The system also includes at least one processor configured to review the physiological data from the plurality of sensors and the indication from the user interface. The processor is also configured to assemble the physiological data into sets of time-series data and analyze the sets of time-series data to determine signature profiles consistent with the administration of at least one drug having anesthetic properties, wherein the signature profiles are determined using signals from the time-series data in an alpha frequency range. The processor is further configured to identify, using signature profiles, at least one of a current state and a predicted future state of the patient based on the indication, and control the administration of the least one drug to attain the predicted future state. The processor is further configured to generate a report indicating at least one of the current state and the predicted future state of the patient induced by the drug.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The present invention will hereafter be described with reference to the accompanying drawings, wherein like reference numerals denote like elements.

FIG. 1A is an EEG waveform illustrating a burst suppression pattern over a 5 minute interval taken from a patient following a propofol bolus induction.

FIG. 1B is an EEG waveform illustrating a corresponding binary signal to the pattern of FIG. 1A where 1 represents a suppression and 0 represents a burst.

FIG. 1C is an EEG waveform illustrating a one minute segment taken from the pattern in FIG. 1A.

FIG. 1D is an EEG waveform illustrating a corresponding binary signal to the pattern of FIG. 1C.

FIG. 2 is a schematic block diagram of a traditional anesthetic compound monitoring and control system that depends completely upon a clinician.

FIG. 3 is a schematic illustration of a traditional closed-loop anesthesia delivery (CLAD) system.

FIG. 4 is an illustration of a close-loop monitoring and control system in accordance with the present invention.

FIG. 5 is a flow chart setting forth the steps of a monitoring and control process in accordance with the present invention, for example, as may be implemented using a system such as described with respect to FIG. 4.

FIG. 6 is a flow chart setting forth steps of a control method in accordance with the present invention.

FIG. 7 is an illustration of a further close-loop monitoring and control system in accordance with the present invention.

FIG. 8 is a flow chart setting forth the steps of an expectation maximization (EM) algorithm in accordance with the present invention.

FIG. 9A is a schematic illustration of a two-compartment pharmacokinetics model in accordance with the present invention.

FIG. 9B illustrates a waveform subjected to a thresholding algorithm in accordance with the present invention to yield a binary waveform for a binary or BSP filter.

FIG. 10 is a block diagram of a closed-loop monitoring and control system in accordance with the present invention.

FIG. 11A is an EEG waveform of a Sprague-Dawley rat during an experiment testing the effect of physostigmine on burst suppression.

FIG. 11B. is a plot illustrating a binary signal associated with the EEG of FIG. 11A.

FIG. 11C is a BSR estimate waveform associated with the EEG waveform of FIG. 11A and computed in one second epochs.

FIG. 11D is a BSP estimate waveform associated with the EEG of FIG. 11A and computed with a binary smoother in one second epochs.

FIG. 12A is a BSP estimate waveform of a Sprague-Dawley rat with confidence intervals computed in one second epochs.

FIG. 12B is a point-by-point BSP comparison matrix.

FIG. 13A is an EEG waveform recorded in a patient undergoing hypothermic arrest.

FIG. 13B is a binary signal associated with the EEG of FIG. 13A.

FIG. 13C is a BSR estimate waveform associated with the EEG of FIG. 13A and computed in one second epochs.

FIG. 13D is a BSP estimate waveform associated with the EEG of FIG. 13A and computed with the binary filter in one second epochs.

FIG. 13E is a BSP estimate waveform associated with the EEG of FIG. 13A after use of the smoothing algorithm.

FIG. 14A is a recorded EEG waveform of a patient following a propofol bolus induction for surgery.

FIG. 14B is a binary signal associated with the EEG of FIG. 14A.

FIG. 14C is a BSR estimate waveform associated with the EEG of FIG. 14A and computed in one second epochs.

FIG. 14D is a BSP estimate waveform associated with the EEG of FIG. 14A and computed with the binary filter in one second epochs.

FIG. 14E is a BSP estimate waveform associated with the EEG of FIG. 14A after use of the smoothing algorithm.

FIG. 15A is a simulated BSP time course after a 10-second bolus of 8 mg kg⁻¹ propofol in the optimized rat two-compartment model.

FIG. 15B is a simulated BSP time course after a 10-second bolus of 3.5 mg kg⁻¹ etomidate in the optimized rat two-compartment model.

FIG. 16A shows the time courses for a bolus dose of propofol in rats illustrating the accuracy of binary or BSP filter as tested on simulated dynamic data.

FIG. 16B shows the time courses for bolus dose of etomidate in rats illustrating the accuracy of binary or BSP filter as tested on simulated dynamic data.

FIGS. 17A-17D′ are simulations at six targets for infusion of propofol 17A-17D and etomidate 17A′-17D′ in rats. FIGS. 17A and 17A′ are an ideal deterministic scenario in which the feedback is the numerically evaluated x₂ₜ transformed to pₜ. FIGS. 17B and 17B′ are a noisy deterministic scenario in which the feedback is the numerically evaluated x₂ₜ transformed to pₜ with Gaussian relative error of standard deviation 3% added. FIGS. 17C and 17C′ are BSP time courses as evaluated from the underlying pharmacokinetics models. Ten simulations at each target were run in the stochastic scenario, in which the feedback is the binary or BSP filter's output, \hat{p}_{t|t}. FIGS. 17D and 17D′ are ten time courses at each target of the observable \hat{p}_{t|t} feedback signal.

FIG. 18A is a simulated BSP time course after a 10-second bolus of 250 mg kg⁻¹ propofol in optimized human two-compartment pharmacokinetic model compared to four-compartment model.

FIG. 18B is a simulated BSP time course after a 10-second bolus of 110 mg kg⁻¹ etomidate in optimized human two-compartment model compared to four-compartment model.

FIGS. 19A-D′ are simulations at six targets for infusion of propofol 19A-19D and etomidate 19A′-19D′ in humans. FIGS. 19A and 19A′ are an ideal deterministic scenario in which the feedback is the numerically evaluated x₂ₜ transformed to pₜ. FIGS. 19B and 19B′ are a noisy deterministic scenario in which the feedback is the numerically evaluated x₂ₜ transformed to pₜ, with Gaussian relative error of standard deviation 3% added. FIGS. 19C and 19C′ are BSP time courses as evaluated from the underlying pharmacokinetics models. Ten simulations at each target were run in the stochastic scenario, in which the feedback is the binary or BSP filter's output, \hat{p}_{t|t}. FIGS. 19D and 19D′ are ten time courses at each target of the observable \hat{p}_{t|t} feedback signal.

FIGS. 20A and 20B are time courses of propofol infusion in the human model simulated with a changing BSP target. FIG. 20A shows the simulated observable stochastic signal with a changing BSP target, namely 0.3, 0.8, 0.6, 0.1, and 0. FIG. 20B shows the infusion rate, as calculated by the proportional-integral controller, corresponding to the variable-target simulation.

FIG. 21A shows photographs of an 8×8 grid array of platinum iridium electrodes. FIG. 21B shows a sagittal maximum-intensity projection of a subdural grid electrode array in a postoperative CT scan. FIG. 21C shows a linear penetrating depth electrode array. FIG. 21D shows a coronal maximum-intensity projection of a depth electrode array in a postoperative CT scan.

FIG. 22 shows example spectrograms of a patient during propofol anesthesia, illustrating region specific changes in alpha power during loss of consciousness.

FIG. 23 shows intracranial electrode coverage for several patients, including one MRI image for each depth electrode array, with electrode locations projected onto a semi-coronal slice aligned to the medial and lateral electrode coordinates of the array.

FIG. 24 shows example spectrograms of another patient during propofol anesthesia, illustrating region specific changes in alpha power during loss of consciousness.

FIG. 25 shows region-specific alpha power changes during induction of propofol during general anesthesia, whereby changes in median alpha frequency power (dB) are overlaid on transparent renderings of a data set average brain.

FIG. 26 is a graphical example illustrating that during propofol general anesthesia, hippocampal alpha oscillations are distinct from anterior alpha oscillations with respect to slow oscillation coupling and presence during suppression periods of burst suppression.

FIG. 27 is a graphical example illustrating a disruption in functional alpha rhythms at loss of consciousness.

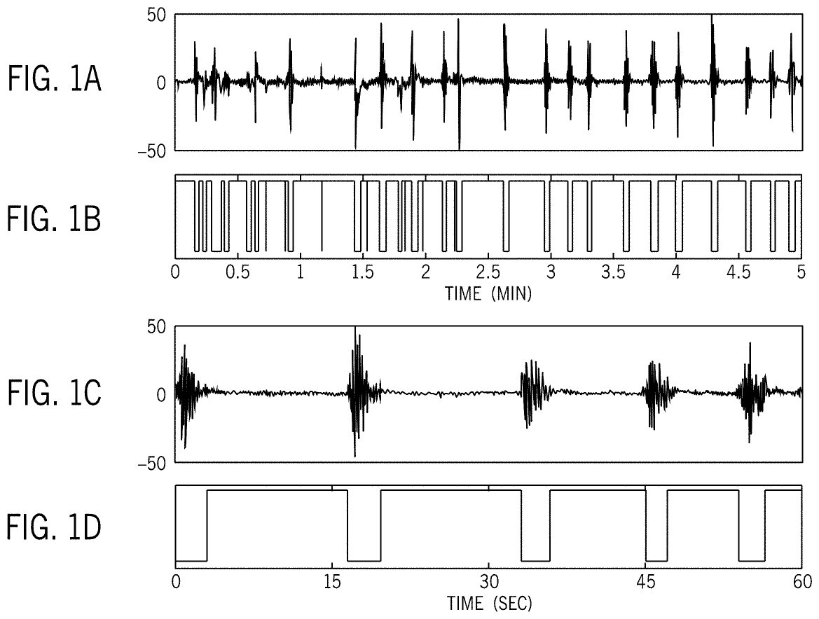

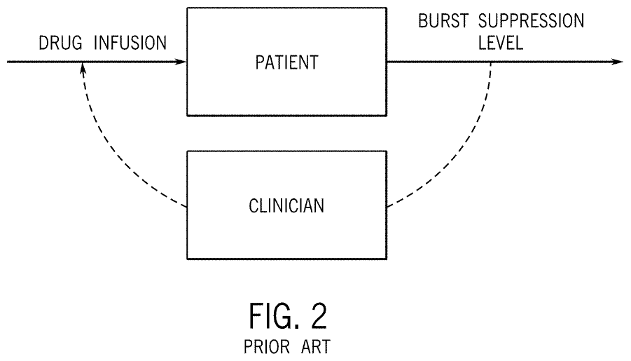

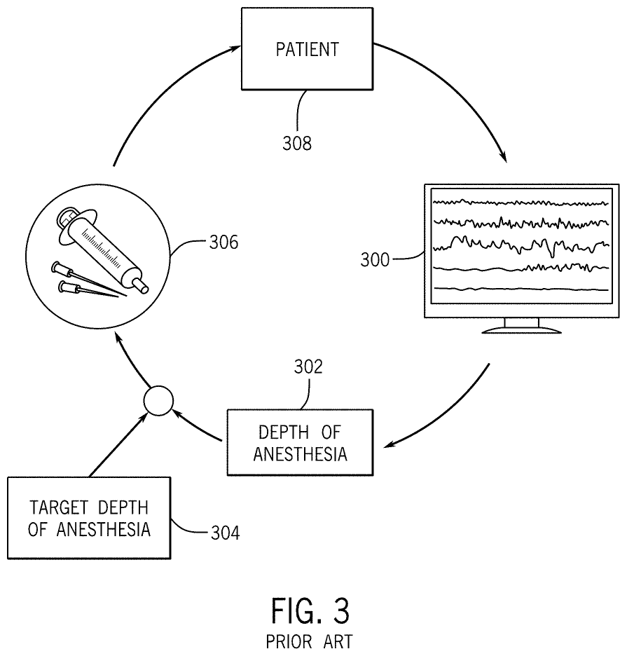

### DETAILED DESCRIPTION

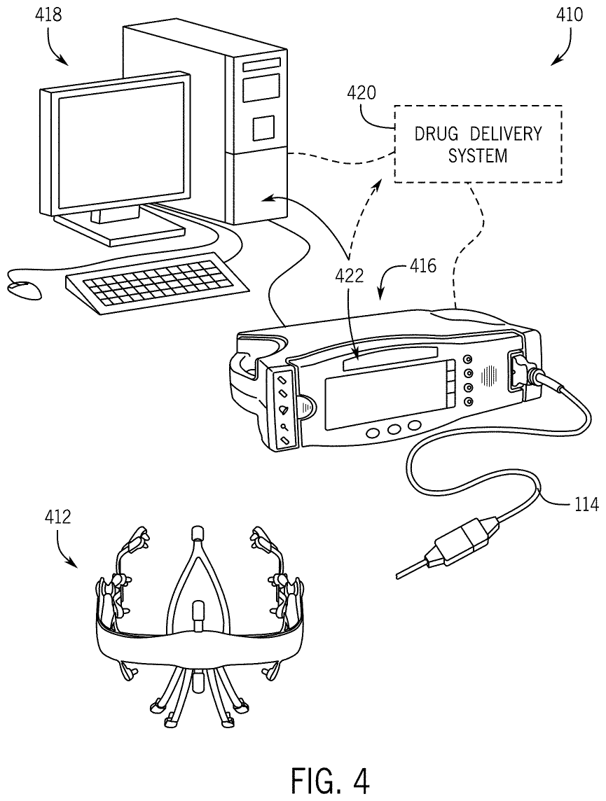

In one embodiment of the present invention, systems and methods for monitoring and controlling a state of a patient during and after administration of an anesthetic compound or compounds are provided. Specifically, referring to FIG. 4, an exemplary system 410 in accordance with the present invention is illustrated. The system 410 includes a patient monitoring device 412, such as a physiological monitoring device, illustrated in FIG. 4 as an electroencephalography (EEG) electrode array. However, it is contemplated that the patient monitoring device 412 may also include mechanisms for monitoring galvanic skin response (GSR), for example, to measure arousal to external stimuli or other monitoring system such as cardiovascular monitors, including electrocardiographic and blood pressure monitors, and also ocular Microtremor monitors. One specific realization of this design utilizes a frontal Laplacian EEG electrode layout with additional electrodes to measure GSR and/or ocular microtremor. Another realization of this design incorporates a frontal array of electrodes that could be combined in post-processing to obtain any combination of electrodes found to optimally detect the EEG signatures described earlier, also with separate GSR electrodes. Another realization of this design utilizes a high-density layout sampling the entire scalp surface using between 64 to 256 sensors for the purpose of source localization, also with separate GSR electrodes.

The patient monitoring device 412 is connected via a cable 414 to communicate with a monitoring system 416. Also, the cable 414 and similar connections can be replaced by wireless connections between components. As illustrated, the monitoring system 416 may be further connected to a dedicated analysis system 418. Also, the monitoring system 416 and analysis system 418 may be integrated.

The monitoring system 416 may be configured to receive raw signals acquired by the EEG electrode array and assemble, and even display, the raw signals as EEG waveforms. Accordingly, the analysis system 418 may receive the EEG waveforms from the monitoring system 416 and, as will be described, analyze the EEG waveforms and signatures therein based on a selected anesthesia compound, determine a state of the patient based on the analyzed EEG waveforms and signatures, and generate a report, for example, as a printed report or, preferably, a real-time display of signature information and determined state. However, it is also contemplated that the functions of monitoring system 416 and analysis system 418 may be combined into a common system.

As will be detailed, the system 410 may also include a drug delivery system 420. The drug delivery system 420 may be coupled to the analysis system 418 and monitoring system 416, such that the system 410 forms a closed-loop monitoring and control system. As will be described, such a closed-loop monitoring and control system in accordance with the present invention is capable of a wide range of operation, but includes user interfaces 422 to allow a user to configure the closed-loop monitoring and control system, receive feedback from the closed-loop monitoring and control system, and, if needed reconfigure and/or override the closed-loop monitoring and control system.

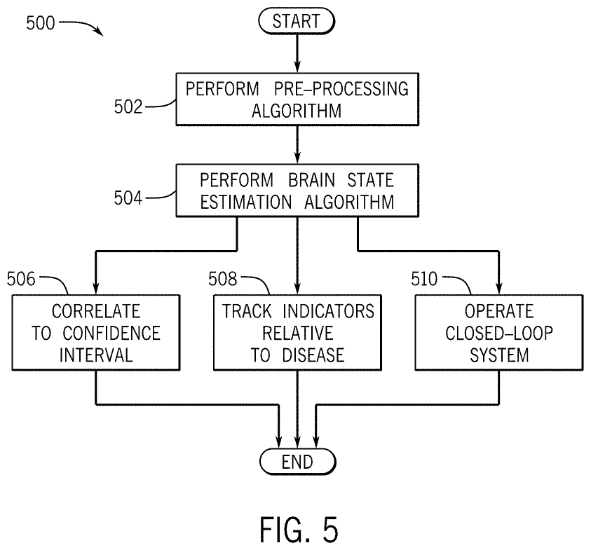

Referring to FIG. 5, a process 500 in accordance with the present invention begins at process block 502 by performing a pre-processing algorithm that analyzes waveforms from an EEG monitoring system. Specifically, at process block 502, identified indicators, such as burst and suppression intervals in the EEG waveform, may be converted into a binary time series. This time series is preferably a “real-time” series that, at process block 504, is provided as an input into a brain state estimation algorithm. One such brain state estimation algorithm is the BSP algorithm, which provides a second-to-second estimate of the brain's state of burst suppression using the concept of a state space model for binary and point process observations.

As will be described in greater detail, the brain state estimation algorithm output, at process block 506, is correlated with “confidence intervals.” The confidence intervals are predicated on formal statistical comparisons between the brain state estimated at any two time points. Also, at process block 508, the output of the brain state estimation algorithm can be used to identify and track brain state indicators, such as burst suppression, during medical procedures or disease states. Exemplary medically-significant states include hypothermia, general anesthesia, medical coma, and sedation to name but a few. The output of the brain state estimation algorithm can further be used, at process block 510 as part of a closed-loop anesthesia control process.

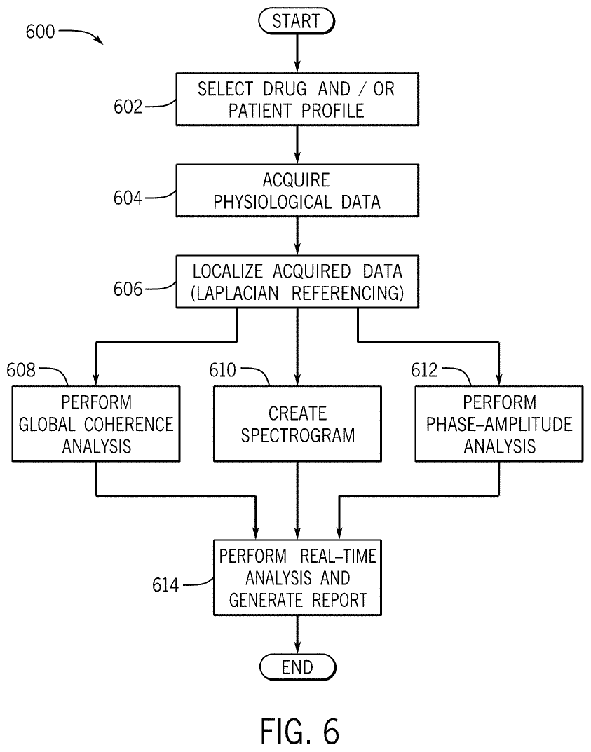

In another embodiment, the present invention provides a system and method for analysis and reporting. Referring to FIG. 6, the process 600 begins at process block 602 with the selection of a desired drug, such as anesthesia compound or compounds, and/or a particular patient profile, such as a patient's age height, weight, gender, or the like. Furthermore, drug administration information, such as timing, dose, rate, and the like, in conjunction with the above-described EEG data may be acquired and used to estimate and predict future patient states in accordance with the present invention. As will be described, the present invention recognizes that the physiological responses to anesthesia vary based on the specific compound or compounds administered, as well as the patient profile. For example, elderly patients have a tendency to show lower amplitude alpha power under anesthesia, with some showing no visible alpha power in the unconscious state. The present invention accounts for this variation between an elderly patient and a younger patient. Furthermore, the present invention recognizes that analyzing physiological data for signatures particular to a specific anesthetic compound or compounds administered and/or the profile of the patient substantially increases the ability to identify particular indicators of the patient's brain being in a particular state and the accuracy of state indicators and predictions based on those indicators.

For example, the following drugs are examples of drugs or anesthetic compounds that may be used with the present invention: Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and the like. However, the present invention recognizes that each of these drugs, induces very different characteristics or signatures, for example, within EEG data or waveforms.

With the proper drug or drugs and/or patient profile selected, acquisition of physiological data begins at process block 604, for example, using a system such as described with respect to FIG. 4, where the acquired data is EEG data. The present invention provides systems and methods for analyzing acquired physiological information from a patient, analyzing the information and the key indicators included therein, and extrapolating information regarding a current and/or predicted future state of the patient. To do so, rather than evaluate physiological data in the abstract, the physiological data is processed. Processing can be done in the electrode or sensor space or extrapolated to the locations in the brain. As will be described, the present invention enables the tracking of the spatiotemporal dynamics of the brain by combining additional analysis tools, including, for example, spectrogram, phase-amplitude modulation, coherence, and global coherence analyses. As will be apparent, reference to “spectrogram” may refer to a visual representation of frequency domain information.

Laplacian referencing can be performed at process block 606 to estimate radial current densities perpendicular to the scalp at each electrode site of, for example, the monitoring device of FIG. 4. This may be achieved by taking a difference between voltages recorded at an electrode site and an average of the voltage recorded at the electrode sites in a local neighborhood. Other combinations of information across the plurality of electrodes may also be used to enhance estimation of relevant brain states. Next, process blocks 608 and 610 yield two pieces of valuable information, namely, the spectrogram and global coherence information, which show different spatiotemporal activity at different states of the patient receiving anesthesia. Though “spectrogram” processing is performed, a visual representation of the spectrogram need not be displayed. In one example, for propofol, when patients are awake, the spectrograms will show strong occipital a activity. After loss of consciousness, the spectrograms will show a loss of a activity and an increase in δ activity, in the occipital sites and strong α and δ activity in the frontal sites. Increased power in the α (8-14 Hz), β (12-30 Hz), and δ (1-4 Hz) ranges in the frontal sites will occur after loss of consciousness, consistent with the well-known pattern of anteriorization. As patients lose responsiveness, the coordinated activity over the occipital sites in the a range diminish. When patients are unconscious, strong coordinated activity in the a range is observed broadly over the frontal electrode sites at which the spectrograms show the anteriorization pattern. Despite the overall high 6 activity in the spectrograms, coordinated activity may only be observed in the a range. The relative power in the occipital α and δ ranges reliably track the patients' behavioral responses. For propofol, the occipital a power is greater than the δ power when the patient is awake, and the reverse is true when the patients are unconscious. The strong global coherence in the α range indicates highly coordinated activity in the frontal electrode sites. Thus, global coherence and weight matrices along with spectrograms provide a first level of data for determining a current state and predicting a future state of a patient's brain under anesthesia. Spectrograms and related coherence and global coherence estimates could be made using the multitaper method to achieve precise and specific time-frequency resolution and efficiency properties necessary to estimate the relevant brain states. Further details regarding initial testing and validation of such processes are provided in Cimenser A, Purdon P L, Pierce E T, Walsh J L, Salazar-Gomez A F, Harrell P G, Tavares-Stoeckel C, Habeeb K, Brown E N (2011). Tracking brain states under general anesthesia by using global coherence analysis. Proceedings of the National Academy of Sciences of the United States of America 108:8832-8837. Other signals may likewise be tracked, such as global coherence and phase-amplitude modulation.

At process block 612, phase-amplitude analysis is performed that considers the amplitude of a given signal with respect to the phase of other signals and vice versa. As explained above, spectral analysis of EEG recordings allows the present invention to track systematic changes in the power in specific frequency bands associated with administration of anesthesia, including changes in δ (1-4 Hz), θ (5-8 Hz), α (8-14 Hz), β (12-30 Hz), and γ (30-80 Hz). However, spectral analysis treats oscillations within each frequency band independently, ignoring correlations in either phase or amplitude between rhythms at different frequencies.

The above-described selection of an appropriate analysis context based on a selected drug or drugs (process block 602), the acquisition of data (process block 604), and the analysis of the acquired data (process blocks 608-612) set the stage for the new and substantially improved real-time analysis and reporting on the state of a patient's brain as an anesthetic or combination of anesthetics is being administered and the recovery from the administered anesthetic or combination of anesthetics occurs. That is, although, as explained above, particular indications or signatures related to the states of effectiveness of an administered anesthetic compound or anesthetic compounds can be determined from each of the above-described analyses (particularly, when adjusted for a particular selected drug or drugs), the present invention provides a mechanism for considering each of these separate pieces of data and more to accurately indicate and/or report on a state of the patient under anesthesia and/or the indicators or signatures that indicate the state of the patient under anesthesia.

Specifically, referring to process block 614, any and all of the above-described analysis and/or results can be reported and, in addition, can be coupled with a precise statistical characterizations of behavioral dynamics. That is, behavioral dynamics, such as the points of loss-of-consciousness and recovery-of-consciousness can be precisely, and statistically calculated and indicated in accordance with the present invention. To do so, the present invention may use dynamic Bayesian methods that allow accurate alignment of the spectral and global coherence analyses relative to behavioral markers.

As stated above, the present invention is not only able to control the administration of anesthetic compounds for the purpose of placing the patient in a state of reduced consciousness influenced by the anesthetic compounds, such as “medical coma,” but can implement and reflect systems and methods for bringing a patient to and from a state of greater or lesser consciousness. For example, co-pending application PCT/US2011/050213, entitled “REVERSAL OF GENERAL ANESTHESIA BY ADMINISTRATION OF METHYLPHENIDATE, AMPHETAMINE, MODAFINIL, AMANTADINE, AND/OR CAFFINE,” is incorporated herein by reference in its entirety.

Prior to the above-referenced work in the co-pending application, efforts utilized the classic approach of developing drugs that antagonize the actions of general anesthetics at the molecular level. However, such efforts have not been feasible because of the lack of clearly defined molecular targets through which general anesthetics induce loss of consciousness. The present invention recognizes, instead, that at the level of neural circuits and systems, there are arousal pathways that can be utilized to actively induce emergence from general anesthesia.

For example, in accordance with one aspect of the present invention, methylphenidate can be used as an inhibitor of dopamine and norepinephrine reuptake transporters and actively induces emergence from isoflurane general anesthesia. Methylphenidate can be used to restore consciousness, induce electroencephalogram changes consistent with arousal, and increase respiratory drive. The behavioral and respiratory effects induced by methylphenidate can be inhibited by droperidol, supporting the evidence that methylphenidate induces arousal by activating a dopaminergic arousal pathway. Plethysmography and blood gas experiments establish that methylphenidate increases minute ventilation, which increases the rate of anesthetic elimination from the brain. These and other findings are included in Solt et al., “Methylphenidate Actively Induces Emergence from General Anesthesia,” Anesthesiology 2011; 115:791-803, which is incorporated herein by reference in its entirety.

With the above as background, the present invention establishes that methylphenidate or other agents can be used to actively induce emergence from isoflurane, propofol, or other general anesthesia by increasing arousal using a control system, such as described above. For example, in addition to the explanation above, Chemali et al., “Active Emergence from Propofol General Anesthesia Is Induced by Methylphenidate,” Anesthesiology 2012; 116:998-1005, which is incorporated herein by reference in its entirety, describes the use of methylphenidate perform active emergence from the use of propofol as a general anesthesia.

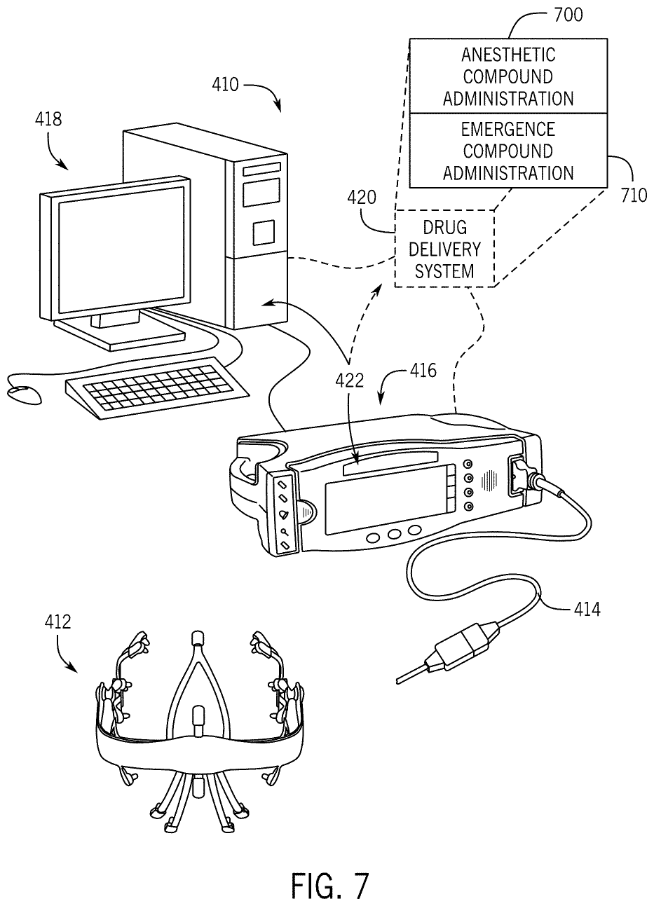

Specifically, a system such as described above with respect to FIG. 4 can be provided to carry out active emergence from anesthesia. That is, such a system can be integrated with the system 410 of FIG. 4. Referring to FIG. 7, the drug delivery system 420 may include two specific sub-systems. Specifically, the drug delivery system 420 may include an anesthetic compound administration system 700 that is designed to deliver doses of one or more anesthetic compounds to a subject, as described in detail above. The drug delivery system 420 may also include a emergence compound administration system 710 that is designed to deliver doses of one or more compounds that will reverse general anesthesia or the enhance the natural emergence of a subject from anesthesia. For example, methylphenidate (MPH) and analogues and derivatives thereof induces emergence of a subject from anesthesia-induced unconsciousness by increasing arousal and respiratory drive. Thus, the emergence compound administration system 710 can be used to deliver methylphenidate (MPH), amphetamine, modafinil, amantadine, or caffeine to reverse general anesthetic-induced unconsciousness and respiratory depression at the end of surgery. The MPH may be dextro-methylphenidate (D-MPH), racemic methylphenidate, or leva-methylphenidate (L-MPH), or may be compositions in equal or different ratios, such as about 50%:50%, or about 60%:40%, or about 70%:30%, or 80%:20%, 90%:10%, 95%:5% and the like. Other agents may be administered as a higher dose of methylphenidate than the dose used for the treatment of Attention Deficit Disorder (ADD) or Attention Deficit Hyperactivity Disorder (ADHD), such as a dose of methylphenidate can be between about 10 mg/kg and about 5 mg/kg, and any integer between about 5 mg/kg and 10 mg/kg. In some situations, the dose is between about 7 mg/kg and about 0.1 mg/kg, or between about 5 mg/kg and about 0.5 mg/kg. Other agents may include those that are inhaled.

In yet another embodiment, a metric or a plurality of metrics are monitored by the system 410, to facilitate accurate monitoring and/or control. For example, as discussed above, one clinically-relevant phenomenon is “burst suppression.” In accordance with one aspect of the present invention, a new state space model has been developed to conduct dynamic analysis of burst suppression. However, instead of traditional metrics for monitoring bust suppression, such as BSR, the present invention introduces the concept of the burst suppression probability (BSP). BSP can be used to interpret the brain's instantaneous likelihood of being in the suppressed state. The aforementioned system 410 may implement a BSP filter algorithm to track burst-suppression in real-time and a smoothing algorithm to analyze burst suppression recorded in a fixed time interval. As will be described, one such approach enables the tracking of burst suppression on a second-to-second time scale and the system 410 can make formal statistical comparisons of this activity at different times.

Specifically, the BSP algorithm, preferably, may be based on a state space framework for point processes and binary observations. The observation model is a binomial process and the temporal evolution of the brain state of burst suppression is defined by a state equation represented as a Gaussian random walk. By making a logistic transformation on the state, the concept of the BSP is introduced to define the brain's state of burst suppression. In accordance with one configuration, the model is estimated using an approximate expectation maximization (EM) algorithm and illustrates its application in the analysis of rodent burst suppression recordings under general anesthesia, a patient emerging from hypothermia, and a patient following induction of general anesthesia. The approach of the present invention obviates the need to artificially average “BSR” over long epochs and allows formal statistical comparisons of burst activity at different time points. The state-space model suggests a more principled and informative way to analyze this important EEG brain state, as will be described in more detail below.

### Brain State Estimation Model

To formulate the state space model in accordance with the present invention, a state space paradigm for analyzing point processes, bust suppression information, and other general binary time series can be used. It can be assumed that EEG data collected over an observation interval (0, T] and the state space model is defined on a discrete set of lattice points within that interval. To define the lattice, a number (l), preferably a large number, is chosen and the interval is divided into l subintervals of equal width Δ=Tl⁻¹. The state space model is evaluated at iΔ for i=1 . . . l.

A state-space model may is characterized by its state and observation equations. The state equation defines the unobservable state process whose evolution we wish to track over time. In one aspect of the present invention, the state represents the brain's state of burst suppression. For example, the state can be defined to be positively related to the probability of suppression. That is, as the state increases the probability of suppression increases and as the state decreases the probability of suppression decreases. The observation equation describes how the observations relate to the unobservable state process. The objective is, thus, to estimate the brain's burst suppression state, burst suppression probability, and their associated confidence intervals.

One may assume that in each interval Δ there can be at most n suppression events. Let bᵢ be the number of suppression events in iΔ. Further assume the observation model is described by the binomial probability mass function as:

$$f ( {b}_{i} \vert {x}_{i} , n ) = ( \begin{matrix}n \\ {b}_{i}\end{matrix} ) {p}^{b} {i ( 1 - {p}_{i} )}^{n - {b}_{i}} , \qquad ( 1 ) ;$$

where pᵢ defined by the logistic function:

$${p}_{i} = \frac{exp ( {x}_{i} )}{1 + exp ( {x}_{i} )} ; \qquad ( 2 )$$

is the BSP and xᵢ is the brain's burst suppression state at time i. In other words, pᵢ is the instantaneous probability of burst suppression. The logistic function links the brain's burst suppression state to the probability of a suppression event and insures that pᵢ remains between 0 and 1 as xᵢ ranges across all real numbers.

The state model may be defined as a random walk:

xᵢ=xᵢ₋₁+∈ᵢ, (3);

where the ϵᵢ are independent Gaussian random variables with mean 0 and variance σ_{ϵ}². This definition of the state provides a stochastic continuity constraint, which insures that the states and, hence, the BSPs that are close in time are close in value. The parameter σ_{ϵ}² governs how rapidly the BSP can change; the larger (smaller) the value of σ_{ϵ}² the more rapidly (slowly) the state and the BSP can change.

To present the estimation algorithm one may take b=(b₁, b₂, . . . , bₗ) and x=(x₁, x₂, . . . , xₗ). Based on the random walk defined in (3), the joint probability density of the state process is:

$$f ( x \vert {σ}_{∈}^{2} , {x}_{0} ) = {( \frac{1}{2 {πσ}_{∈}^{2}} )}^{\frac{I}{2}} exp ( \frac{- 1}{2 {σ}_{∈}^{2}} {\sum }_{i = 1}^{I} {( {x}_{i} - {x}_{i - 1} )}^{2} ) ; \qquad ( 4 )$$

and the joint probability density of the observed suppression events is:

$$F ( b \vert x , n ) = {\prod }_{i = 1}^{I} ( \begin{matrix}n \\ {b}_{i}\end{matrix} ) {p}^{b} {i ( 1 - {p}_{i} )}^{n - {b}_{i}} . \qquad ( 5 )$$

An objective is to estimate using maximum likelihood (ML) the state process x and the parameters σ_{ϵ}² and x₀, where the initial state x₀ is treated as a parameter. Once these estimates are obtained, the BSP can be readily computed with its confidence intervals.

To compute the ML estimates of the parameters we can use an approximate expectation maximization (EM) algorithm for point processes and binary time series. The EM algorithm is a method simultaneously models parameters and an unobservable state process by maximizing the expectation of the complete data log likelihood, doing so by iterating between two steps. In the expectation step, it computes the expected value of the complete data log likelihood given an estimate of the parameters from the previous iteration. In the following maximization step, it computes the parameters that maximize it.

The complete data likelihood is:

$$f ( b , x \vert n , {σ}_{∈}^{2} , {x}_{0} ) = f ( b , x \vert n ) f ( x \vert {σ}_{∈}^{2} , {x}_{0} ) . \qquad ( 6 )$$

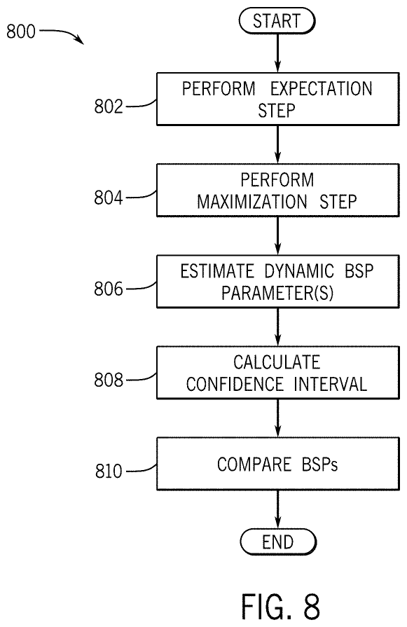

Referring to FIG. 8, one exemplary EM algorithm 800 will be described. At process block 802, an expectation step is performed. The expectation step at process 802 is actually representative of an iterative process. The first iteration starts at a default or otherwise selected position. At iteration l+1, the expected value is computed of the complete data log likelihood given the data b and the estimates T^{(l)} and x₀^{(l)} of the parameters from iteration l using the following:

$$Q ( {σ}_{∈}^{2 ( 𝓁 + 1 )} , {x}_{0}^{( 𝓁 + 1 )} \vert {σ}_{∈}^{2 ( 𝓁 )} , {x}_{0}^{𝓁} ) = E [ log ( f ( b , x \vert n , {σ}_{∈}^{2} , {x}_{0} ) ) \vert \vert b , {σ}_{∈}^{( 2 𝓁 )} , {x}_{0}^{( 𝓁 )} = E [ {\sum }_{i = 1}^{I} log \frac{n !}{{b}_{i} ! ( n - {b}_{i} ) !} + {b}_{i} {x}_{i} - log ( 1 + {e}^{{x}_{i}} ) \vert \vert b , {σ}_{∈}^{( 𝓁 ) 2} , {x}_{0}^{( 𝓁 )} ] + E [ {\sum }_{i = 1}^{I} - \frac{1}{2 {σ}_{∈}^{2}} {( {x}_{i} - {x}_{i - 1} )}^{2} - \frac{I + 1}{2} log ( 2 π ) - \frac{I + 1}{2} log ( {σ}_{∈}^{2} ) - \frac{{x}_{0}^{2}}{{σ}_{∈}^{2}} \vert \vert b , {σ}_{∈}^{2 ( 𝓁 )} , {x}_{0}^{( 𝓁 )} ] . \qquad ( 7 )$$

Expanding the right side of eqn. (7) illustrates the need to estimate three quantities for i=1, . . . , l. The expectation of the state variable conditioned on the data up to time l:

xᵢ|I≡E[xᵢ∥b,σₑ^{2(l)},x₀^{(l)}] (8)

and the covariances of the state variable conditioned on the data up to time l:

W_{i,i|I}≡E[xᵢ²μb,σₑ^{2(l)},x₀^{(l)}] (9); and

W_{i,i-1|I}≡E[xᵢxᵢ₋₁∥b,σₑ^{2(l)},x₀^{(l)}]. (10).

In order to compute these quantities efficiently, the expectation step can be divided into three parts. First, the estimates of x_{i|i}, and σ_{i|i}² are computed using the forward binary or, in accordance with one aspect of the invention, a “BSP” filter, which is a specific implementation of a binary filter. Second, the backward fixed interval smoothing (FIS) algorithm is used to compute x_{i|i} and σ_{i|l}². Finally, state space covariance algorithm is used to compute the covariances W_{i|l}, and W_{i,i-1|l}.

With respect to the binary filter, given the parameters estimates from iteration l, this step estimates x_{i|i} and σ_{i|i}². This means that the step will estimate the state and the variance at i looking at data from the start of the experiment up to i using a non linear recursive forward filter algorithm.

A one step prediction mean and variance are given by:

x_{i|i-1}=x_{i-1|i-1′} (11); and

σ_{i|i-1}²=σ_{i-1|i-1}²+σe₂^{(l)}. (12).

The posterior mode and variance are given by:

x_{i|i}=x_{i|i-1}+σ_{i|i-1}²(bᵢ−np_{i|i}), (13); and

σ_{i|i}²=[(σ_{i|i-1}²)−1+np_{i|i}(1−p)_{i|i})]⁻¹. (14).

The initial conditions are x_{0|0}=x₀^{(l)} and σ_{0|0}²=σₑ²(l). p_{i|i} corresponds to the mode of the posterior distribution. This filter is non-linear because x_{i|i} appears on both sides of (13). It could be calculated recursively using Newton's method, however, when the width of Δ is very small, adjacent states are very close, and we can replace the term p_{i|i} in equation (13) by p_{i-1|i-1}.

With respect to the fixed interval smoother, the posterior mode estimates from the forward filter are used by the FIS to yield the estimates x_{i|l} for i=l−1, . . . , 1. This means that the estimate at time i is conditioned on all the data up to time l. It is a linear filter and the final estimate of the state will thus be a Gaussian distributed variable with mean x_{i|l}, and variance σ_{i|l}². The FIS is:

x_{i|I}=x_{i|i}+Aᵢ(x_{i+1|I}−x_{i+1|i}), (15);

Aᵢ=σ_{i|i}²(σ_{i+1|i}²−1 (16); and

σ_{i|I}²=σ_{i|i}²+Aᵢ²(σ_{i+1|I}²−σ_{i+1|i}²) (17).

The initial conditions are x_{l|l} and σ_{l|l}² previously estimated in the filter algorithm.

With respect to the state space covariance algorithm, σ_{i,j|l} can be derived as follows:

σ_{i,j|I}=Aᵢσ_{i+1,j|I′} (18);

where 1≤i≤j≤l. The covariances are thus given by:

W_{i,i-1|I}=σ_{i,i-1|I}+x_{i|I}x_{i-1|I} (19); and

W_{i,i|I}=σ_{i|I}²+x_{i|I}². (20).

Referring again to FIG. 8, the process 800 continues at process block 804 with the performance of a maximization step. In this context, let T=1/σ_{ϵ}². One may assume that τ has a gamma prior density defined as:

$$f ( τ \vert α , β ) = \frac{{β}^{α}}{Γ ( α )} {( τ )}^{α - 1} exp ( - βτ ) . \qquad ( 21 ) .$$

The complete data log likelihood is maximized with respect to τ using the gamma prior density for T in (21) and then the log posterior is maximized with respect to τ. The expected value of the complete data log likelihood serves as the likelihood in the expression for the posterior.

The log posterior density of T is proportional to:

$$log ( f ( τ \vert α , β ) ) + E [ log [ f ( b , x \vert n , τ , {x}_{0} ) ] \vert \vert b , {τ}^{( 𝓁 )} , {x}_{0}^{( 𝓁 )} = ( α - 1 ) log ( τ ) - βτ + αlog ) ( β ) - log ( Γ ( α ) ) + \frac{I}{2} log ( τ ) - \frac{τ}{2} E [ {\sum }_{i = 1}^{I} {( {x}_{i} - {x}_{i - 1} )}^{2} + 2 {x}_{0}^{2} \vert \vert b , {τ}^{𝓁} , {x}_{0}^{𝓁} ] . \qquad ( 22 )$$

By maximizing (22) with respect to T^{(l+1)} one obtains:

$${}_{τ} ( 𝓁 + 1 ) = {[ I + 2 α - 2 ] [ 2 ( {{\sum }_{i = 2}}^{N} {W}_{i \vert I} - {\sum }_{i = 2}^{N} {W}_{i - 1 , i \vert I} ) + \frac{3}{2} {W}_{1 \vert I} - {W}_{I \vert I} + 2 β ]}^{- 1} . \qquad ( 23 )$$

By maximizing (23) with respect to x₀^{(l+1)} one obtains:

x₀^{(l+1)}=½x_{1|I} (24).

The maximum likelihood estimates of τ or equivalently σ_{ϵ}² and x₀ are respectively τ(∞)=σϵ⁻²(∞) and x₀(∞).

Thereafter, at process block 806, dynamic BSP estimated and, at process block 808, the above-described confidence intervals are calculated. Specifically, the algorithm iterates between the expectation and maximization steps until convergence. The fixed-interval smoothing algorithm evaluated at the ML estimates x₀ and σ_{ϵ}² together with the logistic equation (2) give us the probability of a suppression at time i for i=1, . . . , l. Through a change of variable, the probability density function may be computed which corresponds to the BSP estimate using:

$$f ( p \vert {x}_{i \vert I} , {σ}_{i \vert I}^{2} ) = f ( x \vert {x}_{i \vert I} , {σ}_{i \vert I}^{2} ) \vert \frac{dx}{dp} \vert = [ {( 2 {πσ}_{i \vert I}^{2} )}^{\frac{1}{2}} {p ( 1 - p )}^{- 1} \times exp ( - \frac{1}{2 {σ}_{i \vert I}^{2}} {( log [ {p ( 1 - p )}^{- 1} ] - {x}_{i \vert I} )}^{2} ) . \qquad ( 25 ) .$$

The confidence intervals are obtained by computing the cumulative density of equation (25) and identifying the 2.5th and 97.5th percentiles.

At process block 810, a comparison of BSPs at different times can be performed. Because the logistic transformation that relates the state to the BSP is monotonic, the probability that the BSP at time i is greater than the BSP at time j is obtained by computing the corresponding probabilities of the states. This is done through a Monte Carlo approach. Using the covariance algorithm, for times i and j such that 1≤i<j, the covariance between the augmented state space trials is given by:

$${W}_{i , j \vert I} = {\prod }_{k = i}^{j - 1} {A}_{k} {W}_{j , j \vert I} . \qquad ( 26 )$$

Next, one can then draw M samples from the Gaussian distribution with mean

$$[ \begin{matrix}{x}_{i \vert I} \\ {x}_{j \vert I}\end{matrix} ]$$

and covariance matrix

$$[ \begin{matrix}{W}_{i , i \vert I} & {W}_{i , j \vert I} \\ {W}_{j , i \vert I} & {W}_{j , j \vert I}\end{matrix} ]$$

and count the number G of instances in which the relevant probability is the estimate of Pr(x_{i|l}>x_{j|l}), such that:

$$P ( {x}_{i \vert I} > {x}_{j \vert I} ) = \frac{G}{M} . \qquad ( 27 ) .$$

As described above, the present invention allows the estimation of BSP on a second scale and allows formal statistical comparisons of burst activity at different time points by constructing the confidence intervals of the estimates. The BSP algorithm estimates the joint distribution of the state process, whereby Pr(xᵢ>xⱼ) can be evaluated for any 0≤i<j≤N. This is equivalent to the probability that the BSP at time i is greater than the BSP at time j because the transformation between the state variable xᵢ and the BSP pᵢ is monotonic. The state-space model of the present invention suggests a more principled and informative way to analyze and control this key EEG brain state. Consequently, formal comparisons can be made not only between pre-selected time points but across entire experiments between them and significant changes that occur can be tracked. In one aspect, it may be used for tracking brain states during a medical procedure, such as the administration of an anesthetic or drug, or to assess the progression of disease states, such as a medical coma. In another aspect, it may be used to reveal the dynamical structure of different brain patterns.

### Closed-Loop Monitor/Control Using Burst Suppression

The above-described, the BSP algorithm has wide applicability, for example, in both traditional, clinician-controlled environments, but also when utilizing closed-loop control and drug delivery systems. That is, in one embodiment of the present invention, a closed-loop anesthesia delivery and control system is provided, which may utilize the afore-described BSP algorithm.

The following description of a the closed-loop monitoring and drug delivery control system has wide clinical application. However, for exemplary purposes, the following description will be made with respect to the clinical application of automatic control of medical coma using closed-loop regulation of an anesthetic drug to maintain a specified level of medical coma in terms of a specified level of burst suppression. In this application, the measured level of burst suppression is used as a feedback signal by which the anesthetic infusion is adjusted in a continuous, optimal manner.

While the maintenance of burst suppression is not an objective for general anesthesia, it is a means for providing a stable state of medical coma to aid patients who are recovering from brain injury. For most, drugs are administered at a specified rate, clinical examinations are conducted intermittently, and no continuous EEG tracking of brain state is performed. A common concern with this approach is the eventual overdosing of the drug and the subsequent life-threatening sequelae of these overdose syndromes. It is ironic that the medical coma is a brain state targeted for therapeutic purposes, yet the state of the brain is not generally continuously monitored and controlled. Simulation results provide compelling evidence that maintenance of the brain in a medical coma at a precise level of burst suppression is highly feasible.

In one clinical application, the control target may be to achieve and maintain a target BSR corresponding to medical coma, thereby maintaining an equilibrium effect-site concentration. To design a suitable but not overly-complex model, it may be assumed that, due to the time lag between central compartment drug infusion and effect-site concentration increase, there must be at least two compartments to accurately model the evolution of the drug concentrations. Such a simplified two-compartment model is composed of the central plasma compartment and the auxiliary effect-site compartment connected by a first-order transfer process, ignoring any other peripheral drug distribution compartments.

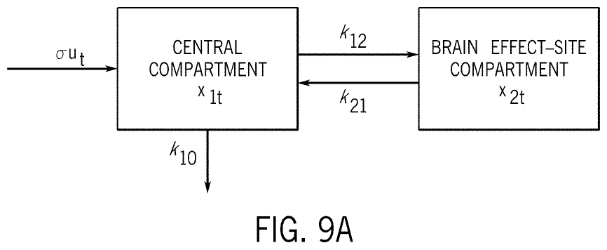

The pharmacokinetic model in FIG. 9A can be described by the second-order linear differential equation system:

$$\frac{dx ( t )}{dt} = Ax ( t ) + I ( t ) \qquad ( 28 ) ;$$

where:

$$\begin{matrix}x ( t ) = [ \begin{matrix}{x}_{1 t} \\ {x}_{2 t}\end{matrix} ] ; & ( 29 a ) \\ A = [ \begin{matrix}- ( {k}_{12} + {k}_{10} ) & {k}_{21} \\ {k}_{12} & - {k}_{21}\end{matrix} ] ; & ( 29 b ) \\ I ( t ) = [ \begin{matrix}σ {u}_{t} \\ 0\end{matrix} ] . & ( 29 c ) ;\end{matrix}$$

and

for an infusion rate of uₜ, in units of μg/s, scaled by a factor of σ. The kinetic rate constants k₁₂ and k₂₁ govern the drug flow between compartments, while the rate constant k₁₀ determines the rate of the drug's clearance from the central compartment. The slow peripheral compartment and fast peripheral compartment, common in many population models, are not included. Once the parameters of this model have been estimated for a subject- and drug-specific model, control simulations can be performed to examine the behavior of the system.

To relate the concentration of the drug in the effect compartment to the propensity of the brain to be in a state of burst suppression, equation (2) may be modified to define the BSP at a given time t as:

$${p}_{t} = \frac{1 - {e}^{- {x}_{2 t}}}{1 + {e}^{- {x}_{2 t}}} . \qquad ( 30 ) .$$

Equation (30) maps the drug concentration in the brain's effect compartment, a number on the interval [0,∞), to a BSP value on [0,1). BSP is a more appropriate term than the BSR defined by Vijn and Sneyd or Rampil and Laster, as it shows explicitly that the index is a number between 0 and 1. Moreover, the BSP leads to a more principled method to relate the EEG to the effect-site-compartment concentration for this problem.

The model in (28)-(30) would be a sufficient starting point from which to design a deterministic controller for a closed-loop control system, provided one could observe either x₂ₜ or pₜ directly. However, this is not the case since only the EEG signal, which is a stochastic process, can be observed. To give a more precise mathematical formulation of the control problem, a stochastic model that relates the EEG to x₂ₜ and pₜ can be defined. To do so, a binary filter algorithm can be implemented, which allows the computation of a dynamic estimate of BSP, \hat{p}_{t|t}, from a thresholded EEG signal and input an error signal in real-time to a system controller.

Turning now to FIG. 9B, an example of how a binary filter may process EEG signals is provided. Thresholding is achieved by differencing the EEG and defining a burst period interval, for example, 100-ms in duration, for which the absolute value of the differenced EEG signal (digitized at any desired frequency, such as, 641.03 Hz) is greater than a threshold (say 15 ρV) at any point of its duration. Similarly, an interval is defined as a suppression period if the absolute value of the differenced EEG signal does not exceed the threshold (say 15 ρV) throughout the interval. Therefore, the thresholded EEG signal may be written as a binary time series defined on any interval t:

$${n}_{t} = {n}_{t} \{ \begin{matrix}0 if the interval is a burst interval \\ 1 if the interval is a suppression interval\end{matrix} . \qquad ( 31 )$$

It may be assumed that the BSP, pₜ, is the probability that nₜ is in the suppressed state. It follows that on any interval t, nₜ is a Bernoulli random variable defined by:

Pr(nₜ)=pₜⁿt(1−pₜ)¹⁻ⁿₜ. (32).

To complete the stochastic model of the EEG and to estimate pt in real-time from nₜ, one may defined define:

zₜ=log(x₂ₜ) (33);

and assume that it obeys the random walk model:

zₜ=zₜ₋₁+vₜ (34);

modified from equation (3), where vₜ is independent Gaussian noise with zero mean and variance σᵥ². Equation (34) is a stochastic continuity constraint that ensures that the updated value of the BSP will be close to the immediately preceding value. The degree of stochastic continuity is governed by σᵥ². The larger (smaller) the value of σᵥ², the greater (lesser) the degree of allowable change in the BSP between adjacent intervals. An estimate for σᵥ² may be taken to be:

$${\hat{σ}}_{v}^{2} = \frac{1}{T} {\sum }_{t = 2}^{T} {( {z}_{t} - {z}_{t - 1} )}^{2} ; \qquad ( 35 )$$

where zₜ denotes the Vijn and Sneyd BSR data transformed using equations (30) and (33). For both models, one may take σᵥ²=10⁵, although other values are possible.

It follows from Chemali J J, Wong K F K, Solt K, Brown E N, “A state-space model of the burst suppression ratio,” IEEE EMBC September 2011, Boston, Mass., which is incorporated herein by reference, that pt may be estimated in real-time from the time series of nₜ by using a binary filter defined for this model as

$$\begin{matrix}{z}_{t \vert t - 1} = {z}_{t - 1 \vert t - 1} ; & ( 36 a ) \\ {σ}_{t \vert t - 1}^{2} = {σ}_{t - 1 \vert t - 1}^{2} + {σ}_{v}^{2} ; & ( 36 b ) \\ {z}_{t \vert t} = {z}_{t \vert t - 1} + \frac{{σ}_{t \vert t - 1}^{2} {c}_{t}}{{p}_{t} ( 1 - {p}_{t} )} ( {n}_{t} - {p}_{t} ) & ( 36 c ) \\ {σ}_{t \vert t}^{2} = {[ \frac{1}{{σ}_{t \vert t - 1}^{2}} + \frac{{c}_{t}^{2}}{{p}_{t} ( 1 - {p}_{t )}} ]}^{- 1} ; & ( 36 d )\end{matrix}$$

where:

$${c}_{t} = \frac{\partial {p}_{t}}{\partial {x}_{2 t}} \frac{\partial {x}_{2 t}}{\partial {z}_{t}} = \frac{{x}_{2 t} {e}^{{x}_{2 t}}}{{e}^{{x}_{2 t}} + 1} ( 1 - {p}_{t} ) . \qquad ( 36 e )$$

Note the modification of equations (11) through (14). Equations (36c) and (36d) are implicit functions in zₜ. To solve, an implementation of Newton's method is used, with stopping criteria:

|z_{t|t}⁽ⁿ⁾−z_{t|t}⁽ⁿ⁻¹⁾|<10⁻⁹ (37a);

and

|f(z_{t|t}⁽ⁿ⁾|<10⁻⁶ (37b).

The function for which a root is desired is:

$$f ( {z}_{t \vert t} ) = - \frac{{z}_{t \vert t} - {z}_{t \vert t - 1}}{{σ}_{t \vert t - 1}^{2}} + \frac{{c}_{t} ( {n}_{t} - {p}_{t} )}{{p}_{t} ( 1 - {p}_{t} )} ; \qquad ( 38 )$$

and due to the small time interval between successive nₜ values, z_{t-1|t-1} is an appropriate initial estimate for z_{t|t} to begin the Newton's method. To reduce computation, the expected value of f′ (y_{t|t}) may be used, since pₜ=E(nₜ):

$$E [ {f}^{′} ( {z}_{t \vert t} ) ] = - [ \frac{1}{{σ}_{t \vert t - 1}^{2}} + \frac{{c}_{t}^{2}}{{p}_{t} ( 1 - {p}_{t} )} ] . \qquad ( 39 ) .$$

Thus, each successive estimate of the root, Z_{t|t}⁽ⁿ⁺¹⁾, may be calculated by a local Fisher's scoring algorithm:

z_{t|t}⁽ⁿ⁺¹⁾=z_{t|t}⁽ⁿ⁾−E[f′(z_{t|t}⁽ⁿ⁾)]⁻¹f(z_{t|t}⁽ⁿ⁾). (40).

Once the stopping criteria are satisfied (which may occur in less than 10 iterations), z_{t|t} and σ_{t|t} are taken to be their estimates from the final iteration. By applying this binary filter to the time series nₜ, one can compute an estimate of pₜ and use it in real-time to compute an error signal for input to a controller.

The stochastic BSP model and binary filter algorithm of the present invention give a principled near-optimal procedure for estimating BSP from the thresholded EEG. Even though \hat{p}_{t|t} is a stochastic signal, because adjacent estimates are separated by milliseconds, this signal is sufficiently smooth and may be used as an input to a deterministic control system.

To simulate a stochastic control problem, the output of the infusion-driven differential equation system is transformed to pt and used at each time interval dt to generate a vector of binary values of length (Fs×dt), where Fs denotes the binary filter's input frequency of nₜ. In one embodiment, a binary filter input frequency of 200 Hz may be used, although other frequencies are possible, with a time interval of 1 second. The binary filter iterates through these integers and generates an updated estimate to the propensity of burst suppression, \hat{p}_{t|t}.

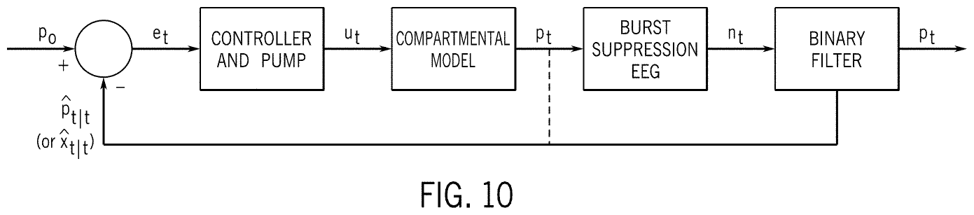

This dynamic estimation of the BSP can be used as negative feedback to generate the error signal that inputs to a proportional-integral (PI) controller. The efficacy of the controller can then be tested in the context of a stochastic feedback signal. Referring to FIG. 10, a closed-loop control can be implemented using a PI scheme, where the control input is calculated as:

uₜ=Kₚeₜ+Kᵢ_{ₜ}Σₑ_{ₜ}. (41).

The signal et is the error defined by:

eₜ=x̅₂−x_{t|t}, (42)

where x̅₂ corresponds to the target BSP. In this scheme, the current state estimation, \hat{p}_{t|t}, is subtracted from the target BSP, p₀, to generate the error signal, eₜ. This error is processed by the controller, which appropriately adjusts the pump's infusion rate, uₜ, to drive the compartmental pharmacokinetics model. The system of differential equations that makes up this underlying system is evaluated numerically to yield x₂ₜ, which is transformed to pₜ. In the deterministic simulation scenarios, this pₜ is taken as the current state estimation, with or without added Gaussian error. In the stochastic simulation scenario, this pₜ is used to generate a sequence of Bernoulli binary integers, nₜ, which are processed by the binary or BSP filter to yield a principled state estimation, \hat{p}_{t|t}.

The feedback signal X_{t|t} is the output of the binary filter. The gains Kₚ and Kᵢ may be chosen in order to achieve suitable performance in terms of the closed loop step-response of the deterministic system, that is, when X_{t|t}=Xₜ. Specifically, it may be advantageous to seek gains that ensure fast rise-time while minimizing overshoot, a potentially undesirable feature in anesthetic induction. A performance specification of this type can be achieved by ensuring that the dominant poles of the closed loop system lie within a prescribed region of the complex plane. In order to complete this procedure, one may use the continuous-time analog of (41) to obtain the open-loop system transfer function:

$$H ( s ) = \frac{{X}_{2} ( s )}{E ( s )} = \frac{( {sK}_{p} + {K}_{i} ) σ {k}_{12}}{s ( s + {k}_{10} ) ( s + {k}_{21} )} . \qquad ( 43 ) .$$

Here, X₂(s) and E(s) are the Laplace transforms of x₂t and eₜ, respectively. It follows from (43) that the poles of the closed-loop system are the solutions of the characteristic equation:

1+H(s)=0. (44).

From (43) and (44) Kᵢ and Kₚ may be selected to affect the location of the closed-loop poles within the s-plane. Given a desire to avoid unnecessary overshoot, one may aim for the fastest dynamics for which the closed-loop poles lie entirely on the real line. In the case of the closed-loop system, it amounts to real and repeated roots of equation (44).

To simplify the analysis, the ratio Kᵢ and Kₚ may be set to:

$$\frac{{K}_{i}}{{K}_{p}} = min \{ {k}_{10} , {k}_{21} \} , \qquad ( 45 ) ;$$

thereby inducing a pole-zero cancellation in (43), yielding:

$$H ( s ) = \frac{{\tilde{X}}_{2} ( s )}{E ( s )} = \frac{σ {k}_{12} {K}_{p}}{s ( s + max \{ {k}_{10} , {k}_{21} \} )} . \qquad ( 46 ) .$$

Substituting this simplified open-loop transfer function into (44), the poles of the closed-loop system are given by the solutions of:

s²+max{k₁₀,k₂₁}s+σk₁₂K=0. (47).

Given the design goal of repeated real roots, one may choose Kᵢ and Kₚ to satisfy:

\sqrt{max{k₁₀,k₂₁}²−4σ−k₁₂Kₚ}=0. (48).

Thus, it follows that the optimal PI controller gains for achieving desired specifications are:

$${K}_{p} = \frac{max {\{ {k}_{10} , {k}_{21} \}}^{2}}{4 σ {k}_{12}} ; \qquad ( 49 )$$

and

Kᵢ=Kₚ min{k₁₀,k₂₁}. (50).

Accordingly, the control gains can be computed in a manner that is specific to the underlying system parameters.

The controller architecture described above is highly robust to parametric uncertainty. This robustness may be characterized in terms of stability margins. In particular, one may assume that the ‘true’ parameters k₁₀, k₂₁ deviate from the model by multiplicative factors σ₁₀ and σ₂₁, respectively. It follows from (43), (49), and (50) that the open loop transfer function is given by:

$$H ( s ) = \frac{{X}_{2} ( s )}{E ( s )} = \frac{{K}_{p} ( s + min \{ {k}_{10} , {k}_{21} \} σ {k}_{12}}{s ( s + {σ}_{10} {k}_{10} ) ( s + {σ}_{21} {k}_{21} )} . \qquad ( 51 ) .$$

Using a standard root-locus argument, it can be verified that the poles of the corresponding closed-loop system are stable for all Kₚ>0 if the following condition holds:

min{k₁₀,k₂₁}<σ₁₀k₁₀+σ₂₁k₂₁. (52);

where a conservative sufficient condition for (52) is:

σ₁₀+σ₂₁>1, (53);

which constitutes a generous tradeoff relationship in parametric uncertainty. For instance, the parameters k₁₀ and k₂₁ would require misidentification by at least 50% in order to compromise the closed-loop stability. Note that this criterion ensures stability for all Kₚ>0, which includes the choice of Kₚ as in equation (49). Moreover, it ensures that arbitrary uncertainty in k₁₂ or σ will not destabilize the system (since such uncertainty enters multiplicatively in Kₚ).

By designing a closed-loop system to have real poles, generous phase margins are ensured, that is, sensitivity to uncertain time-delays. Since (49) and (20) in this approach leads to real-valued poles, these margins may be on the order of 75 degrees. Given that the open-loop system dynamics are relatively slow, this amounts to a tolerance of time delays on the order of minutes.

From (36c) the estimate x_{t|t} may be represented as:

x_{t|t}=xₜwₜ, (54);

where wₜ is a lognormal random variable generated from a Gaussian of zero mean and variance σ_{t|t}². From (14), conceptually this can be viewed as a parametric uncertainty for the feedback gains Kᵢ, Kₚ. However, as shown above, the PI design in (22) and (23) has large stability margins—indeed, Kₚ has infinite gain margin.

Moreover, it is known from the binary filter algorithm that σ_{t|t}² is bounded. It thus follows that the trajectories of the stochastic closed-loop system have finite variance about x̅², a property which has been verified in the Monte Carlo simulation pursued herein.

The above-described systems and methods may be further understood by way of examples. These examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims. For example, specific examples of brain states, medical conditions, and so on, in association with specific drugs and procedures are provided, although it will be appreciated that other drugs, doses, conditions and procedures may also be considered within the scope of the present invention. Likewise, process parameters are recited (for example, signal processing,) that may be altered or varied based on variables such as signal duration, intensity, incidence rate, and so forth.

### Example I

### Physostigmine Effect on Stable Burst Suppression

The following description is with respect to an analysis of a rat under general anesthesia-induced burst suppression The BSP is compared to the BSR in order to illustrate its benefits. For the experiments described herein, signals were first band pass filtered between 5 and 30 Hz. The filtered signals were thresholded and suppression segments less than 500 milliseconds in duration were switched to 1. The binary series was then provided as an input to the BSP algorithm.

The BSP algorithm was evaluated on a rat EEG signal recorded to test whether physostigmine, a cholinergic agonist hypothesized to increase arousal, causes the burst suppression pattern observed during deep anesthesia to switch into continuous activity (associated with increased arousal). In this experiment, it is advantageous to know whether physostigmine, and not saline (control), induces a shift from burst suppression (deep anesthesia) to a delta wave pattern (lighter anesthesia). In case it does, it is desirable characterize the temporal dynamics of that change.

A rat pre-implanted with extradural EEG electrodes was deeply anesthetized with 2% isoflurane. After stabilization of the inhaled concentration, the EEG signal, was recorded for 10 minutes prior to a saline intravenous injection (control). Six minutes later, physostigmine was injected and the recording was maintained for an additional thirty minutes. The total observation interval was of 40 minutes, where the EEG signal was sampled at 512 Hz. The choice of Δ=512 corresponds to a resolution of one second. Both the BSR and the BSP were computed in one second epochs to maximize the detection of dynamics.

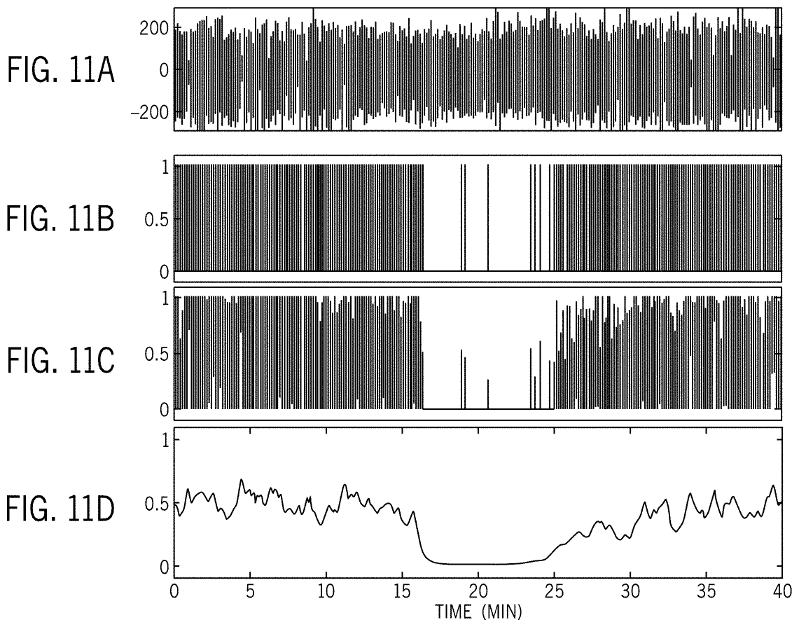

FIGS. 11A-D shows the complete raw signal sampled at 512 Hz (FIG. 11A), the corresponding binary signal (FIG. 11B), the BSR estimates (FIG. 11C) and the BSP estimate (FIG. 11D). At this resolution, the BSR is a very noisy signal that cannot be used for interpretation without further processing. Although there is a clear drop in the BSR at the injection of physostigmine, there is no principled way to measure statistical significance of the drop. Typically, in these experiments, the protocol is repeated in several rats in order to make inferences about the drop.

In contrast, the BSP, using the same resolution, gives a smooth estimate. It is easily seen that the brain state is globally at a stable burst suppression state (BSP is around 0.5). At the injection of physostigmine, an arousal drug, the rat promptly comes out of suppression (BSP is 0) for around 10 minutes at which point the suppression segments reappear and slowly increase to reach back the baseline probability. Because of the fine resolution, the natural fluctuations of the BSP around its mean value are also discerned. For instance, for the BSP between minute 4 and 7, the BSP increases steeply over one minute and then decreases slowly over a few minutes. This means that a long suppression period was then followed by a pattern where burst periods were moderately longer than the suppression periods.

Although the saline injection at minute 16 does not induce any change in dynamics, it is interesting to note the difference in estimates around minute 20 between the BSR and BSP. The BSR is a memory-less measure, which computes the fraction of 1s in each bin without taking into consideration that the EEG signal is a dynamic time-series. Therefore, at minute 18 and 22, the BSR peaks at 0.5 and 0.2 respectively although the overall state clearly does not justify these estimates. In contrast, the BSP estimate consistently remains very close to 0 for this segment. Furthermore, the computed confidence intervals of the estimates allow formal statistical inferences to be made.

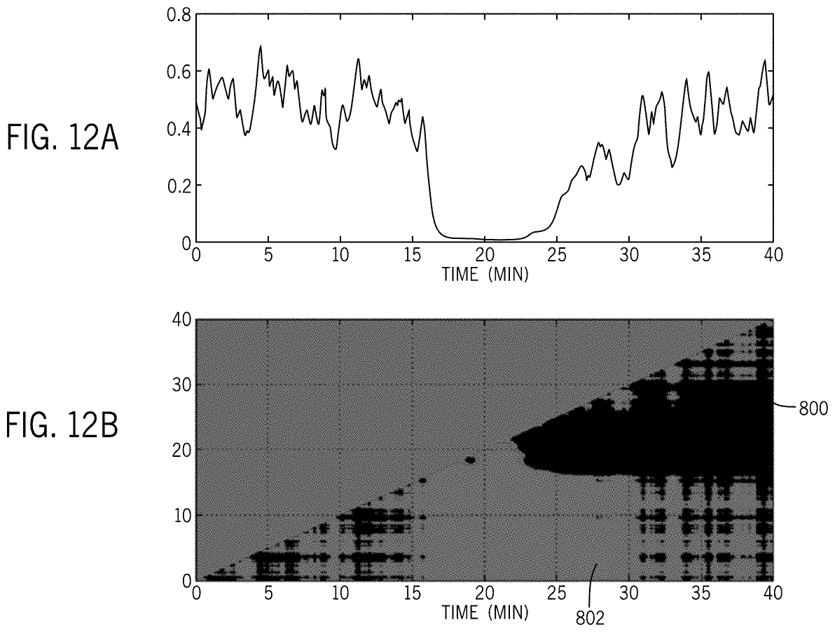

FIG. 12A which shows a BSP estimate waveform of a Sprague-Dawley rat with confidence intervals computed in one second epochs.

FIG. 12B is a point-by-point triangular comparison matrix, showing how every point along the horizontal time axis is compared to all preceding points in time. Evaluating Pr(xᵢ>xⱼ) for the waveform of FIG. 12A, where xⱼ, the y-axis, corresponds to the BSP at time j and xᵢ, the x-axis, corresponds to the BSP at time for l, darker-colored areas 800 illustrate that the corresponding value on the horizontal axis xᵢ is greater than the corresponding value on the vertical axis xⱼ with a probability of 0.95. Similarly, lighter-colored areas 802 illustrate that the corresponding value on the horizontal axis xᵢ is smaller than the corresponding value on the vertical axis xⱼ with a probability of 0.95. In other words, a light-colored entry corresponds to Pr(xᵢ<xⱼ)=0.95 and a dark entry corresponds to Pr(xᵢ>xⱼ)=0.95 where i is the index across the horizontal axis and j is the index across the vertical axis.

It is appreciated from the pattern of light-colored and dark-colored dots in the first 15 minutes and last 10 minutes, that the fluctuations around the mean are not merely explained by a noisy signal, but reflect a significant dynamical structure. If they were not significant, all corresponding entries would have been gray. We also see, that the patterns pre- and post-saline injection are similar.

Moreover, although in this example the change is very dramatic and can be easily seen in the BSP plot, the point-to-point comparison matrix is a confirmation that after the injection of physostigmine at minute 16 until about minute 26 the BSP is significantly smaller than the BSP at all other time points. This abrupt drop in the BSP is expected because physostigmine is a cholinergic agonist that induces increased arousal.

This kind of comparison is advantageous in cases where changes in burst suppression are correlated with a condition of interest. For example, it can be correlated with the progression of a disease such as in subacute sclerosis panencephalitis. In these cases, a measure of significance is fundamental in guiding the diagnosis.

### Example II

### Burst Suppression During Hypothermia

The above-described systems and methods have broad clinical applicability. One exemplary clinical application includes the ability to track of burst suppression during hypothermia. For example, consider the binary filter when used to assess the evolution of the hypothermia induced burst suppression level during a cardiac surgery of around three and a half hours.

The following description is with respect to an analysis of a patient under hypothermia-induced burst suppression. The BSP is compared to the BSR in order to illustrate its benefits. Signals were first band pass filtered between 5 and 30 Hz. The filtered signals were then thresholded and suppression segments less than 500 milliseconds in duration were switched to 1. The binary series was then provided as an input to the BSP algorithm.

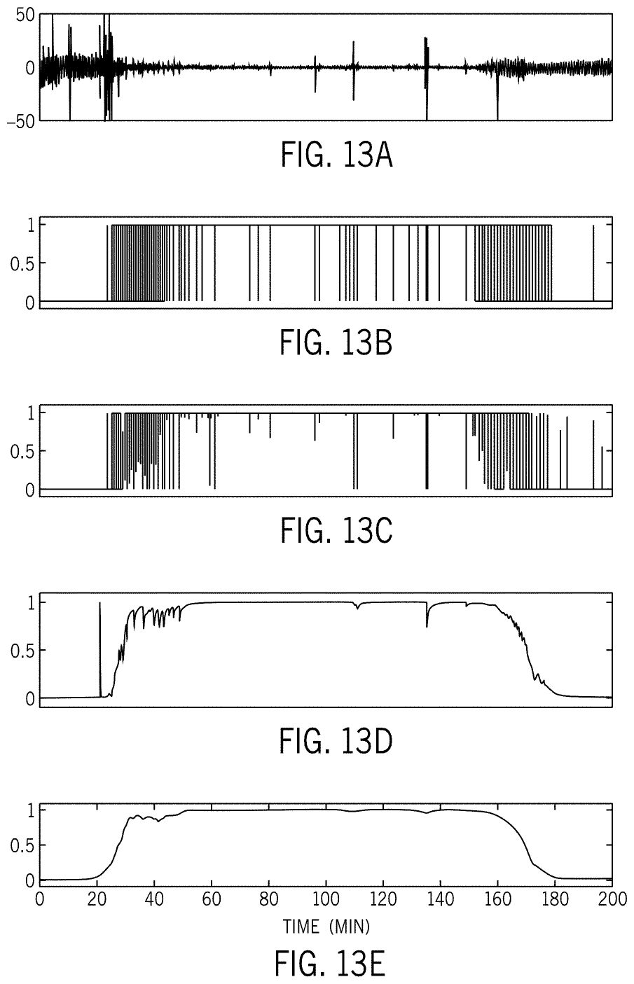

In this example, the EEG signal was recorded from a scalp electrode at the FP1 site referenced to the FZ electrode. The total observation interval was 208 minutes, and the EEG signal was sampled at 250 Hz. The choice of Δ=250 corresponds to a resolution of one second as in the previous case. FIGS. 13A-E show the complete raw signal sampled at 250 Hz (FIG. 13A), the corresponding binary signal (FIG. 13B), the BSR estimate computed in one second epochs (FIG. 13C) the BSP estimate from the binary filter using the ML parameter estimates and (FIG. 13D) the BSP estimate from the smoothing algorithm (FIG. 13E).

The inputs to the binary filter consist of the binary signal, the initial value of the state process x₀, and noise variance σ², which determines how quickly the algorithm tracks the changes in the BSP. In several cases these parameters are readily approximated. The BSR in FIG. 13C shows that after an initial 25 minutes of continuous activity, the EEG enters a burst suppression mode that ceases around minute 50 ceases, turning into an almost isoelectric state. Symmetrically, at about the 150th minute, the burst suppression activity reappears for 25 minutes and the patient comes out of burst suppression. At this resolution, it is very difficult to visually track the level of burst suppression from the BSR.

The binary filter estimate in FIG. 13D is a smoother curve. The general trend of the pattern is very clear, where the increase and decrease are linear, taking around 10 minutes each. The patient remains for about 110 minutes at a stable, almost isoelectric brain state. After the initial increase the ripple in the BSP between minute 20 and 30 indicates that long suppression and long burst periods are present.

The BSP in FIG. 13E is the smoothest curve. It is almost symmetrical between cooling and rewarming. The increase and decrease is linear and takes around 10 minutes in both cases. The patient remains for about 110 minutes at a stable, almost isoelectric brain state.

To use the binary filter, it is possible to define as few as two parameters. Namely, the initial state x₀ and the noise variance σ⁻² can be defined. In several practical cases, these parameters can be confined to a range of realistic values. The binary filter then successfully tracks in real-time the change in burst suppression. By contrast, the BSR is very noisy, and does not give a useful real-time estimate. This suggests that the forward filter is useful when continuous ongoing display of the EEG activity is of interest. Because it can be tracked at very fine resolution, and computes an estimate of the error, it enables the easy and reliable recognition of discrete events.

### Example III

### Burst Suppression During Propofol Induction

Another exemplary clinical application is the tracking of burst suppression during propofol bolus induction. Typically, in the operating room, a bolus dose of an anesthetic is rapidly administered to induce general anesthesia. It is often the case that the patient enters burst suppression within seconds and might remain in that state for several minutes. Since the efficiency of the drug depends on several empirical factors, it is relevant to monitor the level of suppression that is reached and its trajectory, which may help detect any anomaly, or tune the subsequent doses or levels of anesthesia.

The approach of the present invention was evaluated on a burst suppression pattern and its progression induced by a propofol bolus. The EEG was recorded from a scalp electrode at the FP1 site referenced to the FZ electrode of the standard electrode configuration. The total observation interval is of 17 minutes, where the EEG signal was sampled at 250 Hz. The choice of Δ=250 corresponds to a resolution of one second as in the previous case.

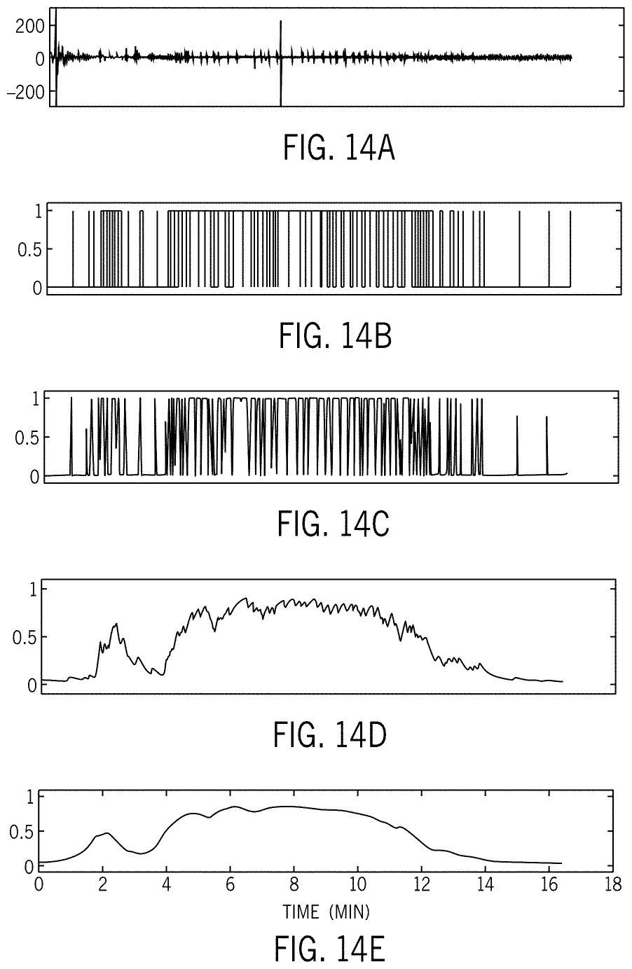

FIGS. 14A-E show the complete raw signal sampled at 250 Hz (FIG. 14A), the corresponding binary signal (FIG. 14B), the BSR estimate computed in one second epochs (FIG. 14C) the BSP estimate from the binary filter using the ML parameter estimates and (FIG. 14D), and the BSP estimate from the smoothing algorithm (FIG. 14E). After the induction, the progression is not monotonous. The filter and smoother BSP climbs to around 0.5 and 0.6 respectively at minute 2, decreases to around 0.2 and 0.1, and then slowly increases to reach a BSP of around 0.7 decreases monotonically to 0. The burst suppression lasts for about 8 minutes, half of which is in an increasing trajectory and the other half in decreasing trajectory.

### Example IV

### Burst Suppression Control During Propofol and Etomidate

Control system identification and formulation was first carried out for propofolnd etomidate infusion in rats. Data collected by Vijn and Sneyd of calculated BSR time courses after 10-second bolus doses (8 mg kg⁻¹ propofol, 3.5 mg kg⁻¹ etomidate) were used for the estimation of pharmacokinetics models. These estimated parameters were used to design the closed-loop controller (see below) in order to yield a critically damped system response, that is, the fastest rise times without overshoot. Table 1 shows the system parameters for both propofol and etomidate infusion in rats.

TABLE 1
The rate constants (given in min⁻¹) and scaling factor for
the pharmacokinetics model estimation and the optimized deterministic
controller gains for the drug-specific rat models.

| | Propofol | Etomidate |
|---|---|---|
| K₁₂ | 0.01557 | 0.01547 |
| K₂₁ | 0.9599 | 0.6703 |
| K₁₀ | 4.552 | 1.993 |
| σ | 0.5885 | 0.8394 |
| Kₚ | 9.423 | 1.275 |
| Kᵢ | 0.1508 | 0.01425 |

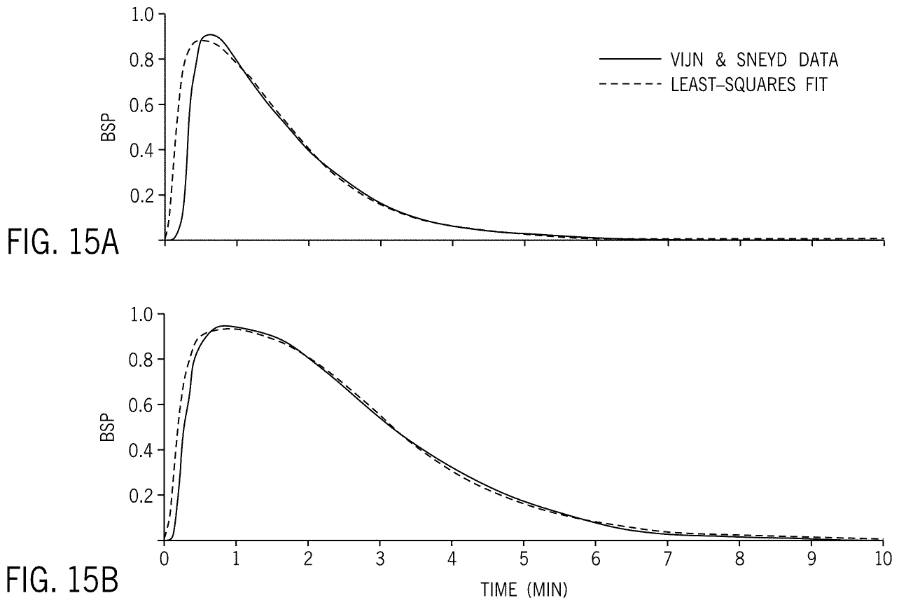

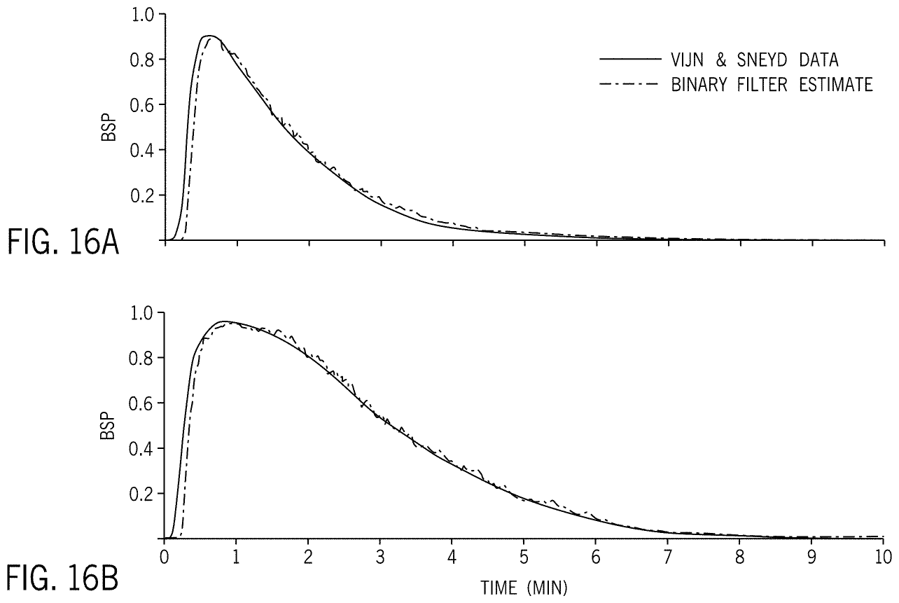

FIGS. 15A and 15B show the simulated BSP time courses for these estimated pharmacokinetic systems compared to the Vijn and Sneyd reported data. To truly test the feasibility of the closed-loop system in accordance with the present invention, the binary filter would need to be added to the closed-loop system to emulate the stochastic nature of its practical application. The algorithm was implemented and tested on the Vijn and Sneyd data to ensure it was accurately outputting a dynamic estimate of pₜ. At each 1-second interval of the Vijn and Sneyd data, a Bernoulli process of 200 binary integers was generated, simulating an EEG signal partitioned into intervals and thresholded. This vector was used as input to the binary filter algorithm, which output one updated \hat{p}_{t|t} per second. Based on the ability of the algorithm to accurately and dynamically estimate the Vijn and Sneyd BSR curve, as is evident in FIGS. 16A-B, it was incorporated into the control loop to model a stochastic control system.

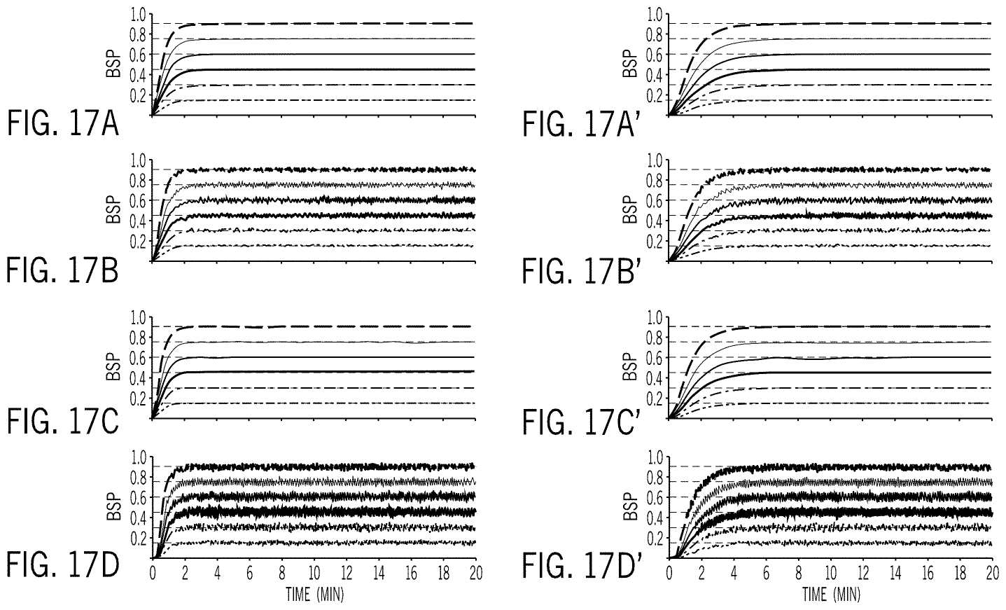

The optimized controller was tested at six target BSP values, equally spaced on the interval [0.15, 0.9]. First, the system response was simulated with a deterministic feedback signal; the numerically evaluated x₂ₜ was simply transformed to pt and fed back at each time interval, without the use of the binary filter. For these six targets, the simulated closed-loop system performed with an average rise time (t_{90%}−t_{10%)} of 1.319 minutes for propofol infusion and 3.031 minutes for etomidate infusion and, as predicted by the controller design theory, no overshoot in either model. To provide a more realistic simulation in this deterministic feedback scenario, Gaussian noise was added to the evaluated x₂ₜ before transformation, with 0 mean and a standard deviation of 3% of its current value. The control system was still able to achieve and maintain a target BSR level with added noise. Finally, the control system was simulated with a stochastic feedback signal by using the binary filter to provide a dynamic estimation of BSP, \hat{p}_{t|t}. FIGS. 17A-D′ show the system response of the rat models in these three feedback scenarios.

Control system identification and formulation was then carried out for propofol and etomidate infusion in humans. Table 2 shows the system parameters for both propofol and etomidate infusion in humans.

TABLE 2
The rate constants (given in min⁻¹) and scaling factor for
the pharmacokinetics model estimation and the optimized deterministic
controller gains for the drug-specific human models.

| | Propofol | Etomidate |
|---|---|---|
| K₁₂ | 0.0001457 | 0.00006598 |
| K₂₁ | 0.8193 | 0.6434 |
| K₁₀ | 0.8236 | 0.4982 |
| σ | 0.1095 | 0.3614 |
| Kₚ | 177.2 | 72.34 |
| Kᵢ | 2.420 | 0.6006 |

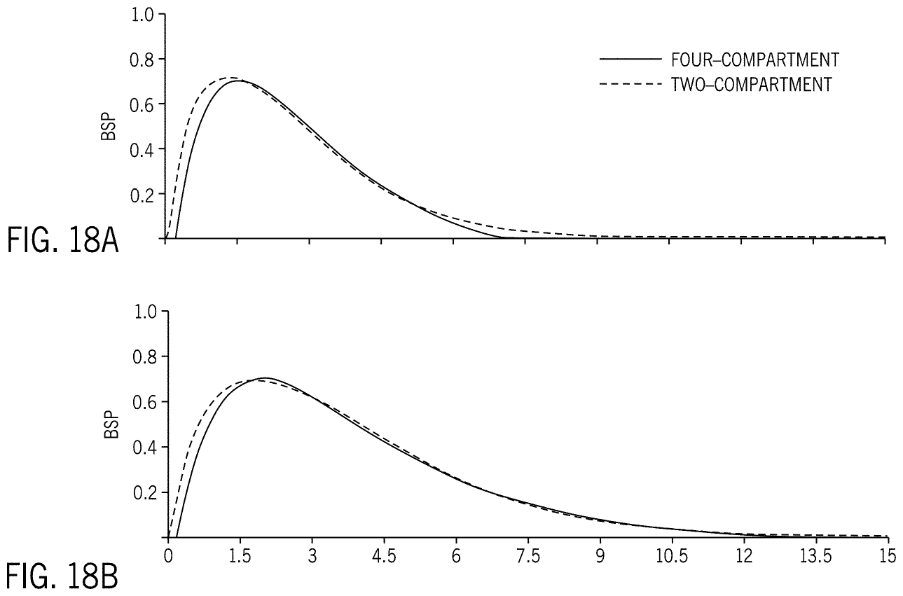

Existing four-compartment pharmacokinetic models were used to generate human BSP time courses analogous to the Vijn and Sneyd data. From these curves, the above-described two-compartment models were fit, and the resulting parameters were used to design the closed-loop controller (see below). FIGS. 18A-B shows these simulated two-compartment model BSP traces compared to the simulated four-compartment model BSP traces.

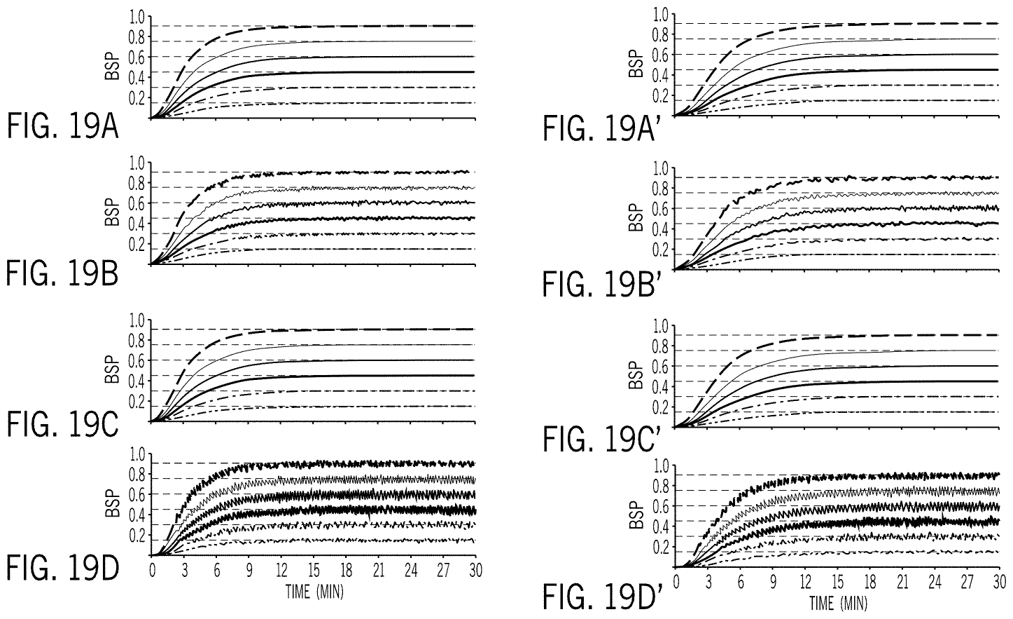

Reasoning that the binary filter would behave exactly the same in the human models as in the rat models, the optimized controller was tested with the human pharmacokinetic models at the same six targets on the interval [0.15, 0.9]. The closed-loop system was again simulated in three feedback scenarios: deterministic feedback of pₜ, deterministic feedback of pt with Gaussian noise, and stochastic binary filter feedback of \hat{p}_{t|t}. With an ideal deterministic feedback signal, our system performed with an average rise time of 7.356 minutes for propofol infusion and 9.411 minutes for etomidate infusion, with no target overshoot. FIG. 19A-D′ shows the system response of the human models in these three feedback scenarios.

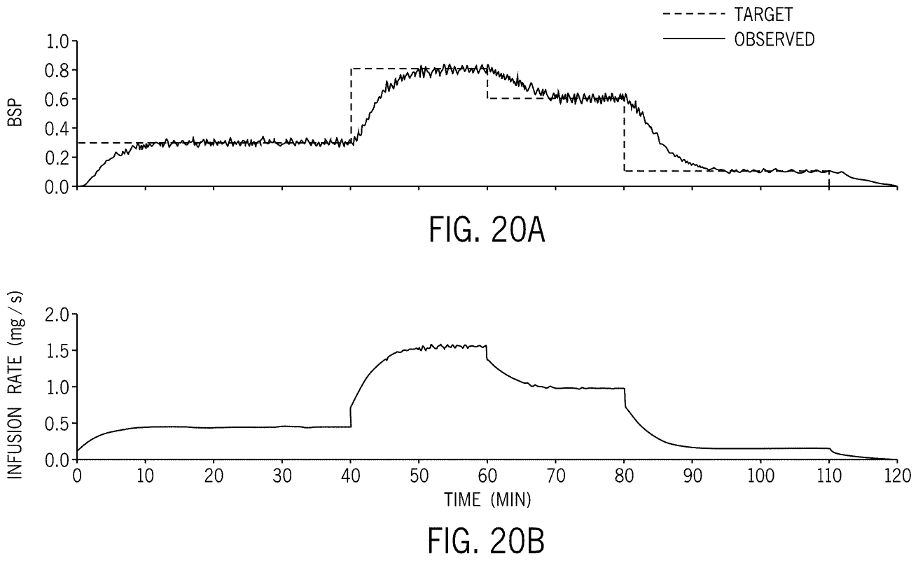

Finally, to simulate the BSP-targeting scheme that could be applied in the clinical setting, the closed-loop system was tested with a changing target, driving the human model deeper into burst suppression, and ending with the patient coming out of burst suppression (see FIG. 20).

With respect to the pharmacokinetics model estimation, data for the rat models was taken from the CLAD system study of Vijn and Sneyd, which presented smoothed time courses of calculated BSR in rats after a 10-second bolus dose (8 mg kg⁻¹ propofol, 3.5 mg kg⁻¹ etomidate). For the human models, existing population pharmacokinetic models were calibrated for a 30-year-old, 180-cm, 70-kg male. For propofol, the population model of Schnider T W, Minto C F, Gambus P L, Andresen C, Goodale D B, et al. (1998) The inuence of method of administration and covariates on the pharmacokinetics of propofol in adult volunteers. Anesthesiology 88: 1170-82, which is incorporated herein by reference, was used to numerically evaluate the effect-site concentration during and after a 10-second bolus of 250 mg kg⁻¹. Since a patient may not enter a state of burst suppression the instant the effect-site concentration is nonzero, the concentration trace was shifted negatively and transformed to pt such that the patient peaked at a BSP of 0.7, and came out of burst suppression 7 minutes after the initiation of the dose. For etomidate, the population model of Arden J R, Holley F O, Stanski D R (1986) Increased sensitivity to etomidate in the elderly: initial distribution versus altered brain response. Anesthesiology 65: 19-27, which is incorporated herein by reference, was used to numerically evaluate the effect-site concentration during and after a 10-second bolus of 110 mg kg⁻¹. The concentration trace was shifted negatively and transformed to pt such that the patient peaked at a BSP of 0.7, and came out of burst suppression 12 minutes after the initiation of the dose.

To fit a two-compartment pharmacokinetic model to this data, a Matlab function was created to calculate the mean squared error between these BSP time courses and a BSP time course numerically evaluated from a given set of two-compartment-model parameters k₁₀, k₁₂, k₂₁, and σ. Matlab's Optimization Toolbox was used to optimize these four parameters of the pharmacokinetic model to minimize this mean squared error, and yield an estimate of the best-fit rate constants and scaling factor.

### Example V

More than 25 million Americans receive general anesthesia (GA) each year and stereotyped signatures of general anesthesia in the electroencephalogram (EEG) have been known since the 1930's. One such signature is a change in the distribution of alpha (8-13 Hz) power in the electroencephalogram from a posterior distribution in the awake state to an anterior distribution in the unconscious state. The intracranial correlates of this effect observed in EEG are not well understood. This study examined neural recordings from 14 patients who had been previously implanted with intracranial depth electrodes, some of whom also had EEG surface electrodes, while those patients were induced with propofol general anesthesia. We show that anteriorization of alpha power during general anesthesia is associated with two distinct phenomena.

The first phenomenon is a disruption of traditional waking alpha oscillations which include the occipital, sensorimotor, and auditory alpha rhythms. We demonstrate that two of the rhythms—sensorimotor and auditory—are related to task in the data set and are disrupted at loss of consciousness. The second phenomenon is of the onset of de novo alpha oscillations in frontal and midline structures including the cingulate cortex, hippocampus, and frontal white matter. We provide evidence of distinct generators for hippocampal and frontal alpha rhythms during general anesthesia.

While, in general anesthesia, EEG alpha dynamics may be a reliable marker of loss of consciousness, in the waking state, alpha dynamics are functionally correlated with sensorimotor behavior, cognition, vision and sleep. Three distinct alpha rhythms have been observed in recordings from the human cortex. The occipital or traditional alpha rhythm is recorded from occipital cortex and is suppressed when the eyes are open. The sensorimotor mu or wicket rhythm is recorded from somatomotor cortex and is suppressed during sensorimotor execution or preparation. The third or tau rhythm is recorded from over a broad region of temporal cortex that includes auditory cortex, and is thought to be suppressed during auditory or cognitive stimuli. Tau cannot readily be identified in surface EEG and must be recorded from intracranial electrodes. These three rhythms are distinct in their distribution over cortex, frequency content, task responsiveness, development in mammals, and relationship with disease states.

Suppression of alpha power during sensation, imagery, planning, and execution during sensorimotor tasks is a general principle of the phenomenology of this oscillation in its three forms. Alpha rhythms during GA, as well as wakefulness, sleep and coma, are thought to occur as a result of neuronal activity in both thalamo-cortical and cortico-cortical networks. A computational model of alpha frequency dynamics with the anesthetic agent propofol (2,6-di-isopropylphenol) suggests that these rhythms are mediated by thalamo-cortical circuits, with propofol strengthening reciprocal projections between cortical pyramidal cells and thalamocortical relay neurons. It has been proposed that localized patterns of alpha band changes during GA may be the result of differential effects of propofol on distinct thalamic nuclei. The prediction of increased anterior alpha power and coherence during unconsciousness have been confirmed in high density EEG studies. Such studies have also established the result that alpha and slow (0.1-1 Hz) frequency EEG dynamics may be tightly coupled after propofol, and that the phase of this relationship may vary systematically with anesthetic depth. However, the mechanisms of alpha and slow frequency coupling are not well understood.

In this report, we examine intracranial neural recordings in a set of human patients, some of which have surface EEG, to establish neurophysiological correlates of alpha power anteriorization during the transition to unconsciousness with propofol. These human subjects have been previously implanted with intracranial electrodes for management of intractable epilepsy. Extending the results of previous research that has been performed with subdural electrode arrays resting on the cortical surface, the electrodes in this data set include depth electrode arrays penetrating into cingulate cortex, hippocampus, and medial white matter. This allows recordings from cortical and subcortical regions that are distant from surface EEG and may be related to the behavioral components of GA.

We hypothesize that anteriorization of EEG alpha power is associated with disruption of the three dominant alpha band rhythms in human cortex: traditional occipital alpha, sensorimotor mu and temporal tau. Moreover, we hypothesize that anteriorization is associated with de novo alpha dynamics in anterior brain regions that do not have a dominant EEG alpha rhythm observable in the waking or sleep states. Previous research has pointed to a role for anterior cingulate cortex as a site of anesthesia induced PET activation changes. The frequency specific effects of propofol in anterior white matter, prefrontal cortex, cingulate cortex, and subcortical regions including hippocampus are not known. We examine power dynamics in the alpha frequency band at these recording sites, and discuss the implications for systems and network level mechanisms of general anesthesia.

### Materials and Methods

Data Collection.

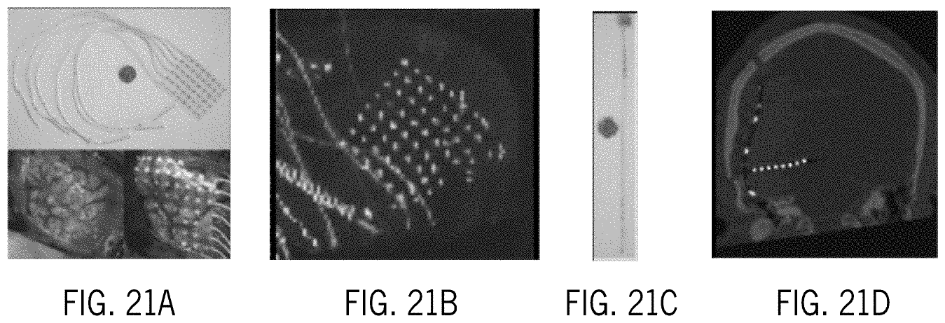

14 patients were implanted with iEEG electrode arrays as part of standard clinical treatment for intractable epilepsy. The arrays included linear penetrating depth arrays having 6-8 electrodes, subdural grid arrays having 16-64 electrodes, and/or subdural strip arrays having 4-16 electrodes (Adtech Medical, Racine, Wis.) (FIG. 21). Surface EEG electrodes were additionally applied to six patients in a subset of the standard 10-20 EEG configuration. Electrode placement was selected by the patients' clinicians without regard to the current study. Patient demographic and clinical information are provided in Table 1. All patients gave informed consent in accordance with protocols approved by the hospital's Institutional Review Board. Recordings were obtained during electrode explant surgery that occurred after 1-3 weeks of inpatient monitoring to determine epileptogenic foci. Signal acquisition began prior to induction of general anesthesia and continued until the electrodes were disconnected for explant. EEG and iEEG signals were recorded with a sampling rate of a 2000, 500, or 250 Hz depending on settings of the hospital's clinical acquisition system. Signals were digitized with hardware amplifiers (XLTEK, Natus Medical, Inc., San Carlos, Calif.) that bandpassed between 0.3 Hz and the sampling rate, and were stored on a computer (Dell) for offline processing. A linked earlobe electrode (A1-A2), a C2 reference on the back of the neck, or an inverted disc electrode on the inner skull table were used as a reference when available; otherwise an average reference was used (N=1, patient 13).

Anesthesia.

All patients underwent induction of general anesthesia with propofol. 13 patients received a bolus dose; one patient received an infusion (patient 2). Drug protocols were selected by the patients' clinicians without regard to the current study.

Behavioral Task.

Patients were delivered auditory stimuli through headphones (prerecorded words and the patient's name) approximately every 4 seconds during the task, and were instructed to respond with a button click. Responses were recorded using stimulus presentation software (Presentation, Neurobehavioral Systems, Inc., Albany, Calif., or EPrime, Psychology Software Tools, Inc., Sharpsburg, Pa.). Loss of consciousness time (LOC) was defined as the time of the first stimulus to which the patient did not respond. One patient was excluded from performing the auditory task by request of his clinician (patient 10). LOC was defined marked at 30 seconds after propofol bolus dose for that patient for display in exemplar figures. This patient was excluded from group analyses of peri-LOC dynamics.

Electrode Localization.

A postoperative CT scan and preoperative T1-weighted MRI scan were obtained for each patient. Data were processed using open-source software (Freesurfer, http://surfer.nmr.mgh.harvard.edu/fswiki) and custom programs written in MATLAB (The Mathworks Inc., Natick, Mass.). Coregistration of postoperative CT to preoperative MRI was computed using automated routines in Freesurfer and verified visually. RAS coordinates were identified for all iEEG electrodes in the subject's anatomical space by visual inspection of a maximal intensity projection of the CT (FIG. 21). Those coordinates were projected to the subject's preoperative MRI space using coregistration matrices. An automatic rendering of the cortical surface was created from the preoperative MRI image. RAS coordinates of electrodes from subdural grid and strip arrays were mapped to the closest coordinate on the rendered cortical surface using a minimum energy procedure. Average cortical surface renderings and MRI volumes were computed as well as transformation matrices between each patient's coordinate system and the group average coordinate systems.

Anatomical Mapping.

Electrode coordinates were automatically mapped to anatomical labels as described in. Cortical parcellation labels were used for grid and strip electrode arrays and volumetric segmentation labels were used for depth electrode arrays. Segmentation and parcellation results were examined visually on the MRI image at each electrode location and spurious results were removed from the data set (n<5%). Functional segmentations were determined from a subset of the structural segmentations, including functional segmentations for the primary auditory cortex, primary somatosensory cortex, primary motor cortex, cingulate cortex, hippocampus, and white matter. Remaining electrodes were segmented into broader anatomical regions due to a smaller number of electrodes outside of those regions previously listed. Occipital, parietal, and inferotemporal cortex were combined, and frontal and orbitofrontal cortex were combined, and temporal cortex (non-auditory) was labeled. These subdivisions were selected to include only grey matter. Unsegmented regions as well as subcortical regions comprising fewer than three channels in the data set were excluded from further analysis.

Data Exclusion.

Individual electrodes with recordings predominated by artifacts (absent signal or amplitude >10× the array median) were excluded from analysis by visual inspection. Shorter segments of data in the remaining electrodes were excluded using the same criteria. Individual electrodes or shorter segments of data were excluded that contained epileptiform discharges, determined by visual inspection. Total time of removed segments was <5%. In one patient, 78/80 channels were removed due to generalized epileptiform discharges (patient 44). In one patient, 16 channels were removed due to the appearance of dysplastic cortex in the MRI (patient 8).

Data Analysis.

In each subject, two epochs were distinguished over the recording period. The preinduction epoch began at a period of time 400 to 150 seconds prior to loss of consciousness. The start time for this epoch was chosen such that the preinduction recording time was approximately 150 seconds in most channels when large recording artifacts were removed. The preinduction epoch ended at the time of the first dose of propofol. We used visual inspection to identify the postinduction epoch. This epoch defined a period of stationary spectral power that occurred after characteristic paradoxical excitation and prior to burst suppression in the five patients who underwent burst suppression. Burst suppression intervals were excluded to avoid the confound of low-power suppression intervals in group analyses, which differed in total time across patients. The post-induction epoch ended at any of these events: a) the first suppression period apparent in the median spectrogram, b) the delivery of any anesthetic drug besides propofol, and c) the end of the recording. The stationary post-induction epoch was identified by visual inspection of the median spectrogram computed across all channels, was identified prior to further analyses, and was verified by an anesthesiologist (E.B.1). The epochs are indicated in the exemplar figures for each patient.

Retained signals were low-pass filtered at 100 Hz and resampled at 250 Hz using finite impulse response filters, and spectrograms were computed for each channel with Chronux software. Spectrograms were computed with 3 tapers, 2 second windows, 1 Hz frequency resolution, and 0.2 second time steps. For display, raw time-series were lowpass filtered below 40 Hz using finite impulse response filters. In all epochs, the median log spectral power between 8-13 Hz was computed for each electrode as the median over spectrogram windows in the epoch and mean over frequency bins in the alpha band.

A modulation index was computed to describe the relationship between slow oscillation (0.1 to 1 Hz) phase and the alpha oscillation amplitude for all channels. The index was computed with 12 phase bins, 10 seconds of time in each phase bin. The analytic phase value extracted using a Hilbert transformation of a signal bandpassed using FIR filters of length 4500 with passbands of 7.5-13.5 Hz for the alpha band, and 0.1-1 Hz for the slow band, with transition bandwidths of 10% or 0.5 Hz, whichever was smaller. To display alpha band amplitude concurrently with an iEEG trace by coloring the trace relative to amplitude, alpha amplitude was normalized to a percentile of the amplitude at all time points over the time period of the displayed trace.

To ascertain task-related modulations of alpha power in each recorded channel, an event related spectral perturbation (ERSP) was computed for each channel in which amplitude in the alpha band was related to auditory stimuli both before and after LOC and button presses prior to LOC. Alpha amplitude was computed as described above in order to have a metric with temporal resolution the same as the sampling rate. A window of [−0.75 to 1.5] seconds was computed around each event time, with the first 0.5 seconds of the window assigned as a baseline. Each window was normalized by removing its mean. Mean normalized alpha amplitude was then computed for each point across the 2.25 second window, and values that was significantly different from the mean amplitude in the baseline window were ascertained for each time point in the window. Significance was computed using 500 iterations of a surrogate control of shuffled times perturbed uniformly over +/−4 seconds. A significance level of alpha=0.05 was computed obtained at every point in the perievent window outside of the baseline and a familywise error rate was used to correct for multiple comparisons. No corrections were made over multiple electrodes.

### Results

Between 54 and 124 channels were recorded in each patient. Intracranial neural recordings were obtained at 1521 recording sites from 14 patients. Seven of those patients also had surface EEG. Five subjects demonstrated burst suppression EEG.

### Characteristic Waking Alpha Rhythms are Abruptly Suppressed and De Novo Alpha Rhythms Emerge at LOC

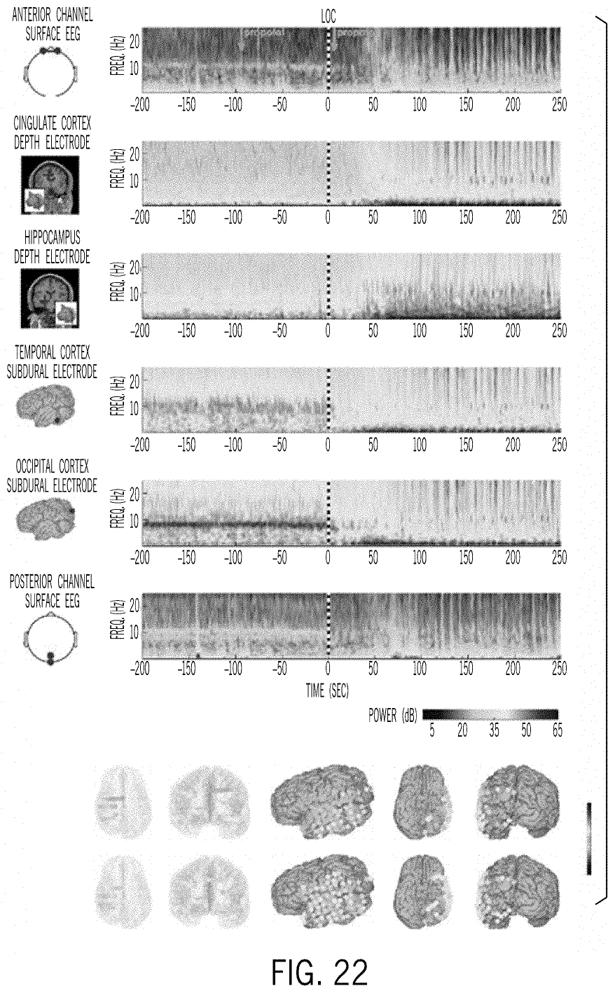

FIG. 22 shows spectrograms of iEEG signals (right panel) that were recorded in several cortical and subcortical regions (localization in left panel) for Patient 9, who had concurrent iEEG and surface EEG. Prior to LOC, an alpha rhythm is observed in the posterior bipolar surface EEG channel, which is simultaneous with an iEEG alpha rhythm recorded in the occipital cortex subdural electrode. This oscillation is consistent with the traditional occipital alpha rhythm because of its spatial location, high power relative to other frequencies and channels, and frequency content. In this patient, a temporal alpha oscillation occurs also in the medial temporal cortex that is spatially consistent with a tau rhythm and occurs with a greater peak frequency in the alpha band over the preinduction epoch (8.7 Hz in the occipital channel and 10.1 Hz in the temporal channel), which may indicate distinct rhythms. Alpha power in both channels is suppressed abruptly within 10 seconds after LOC.

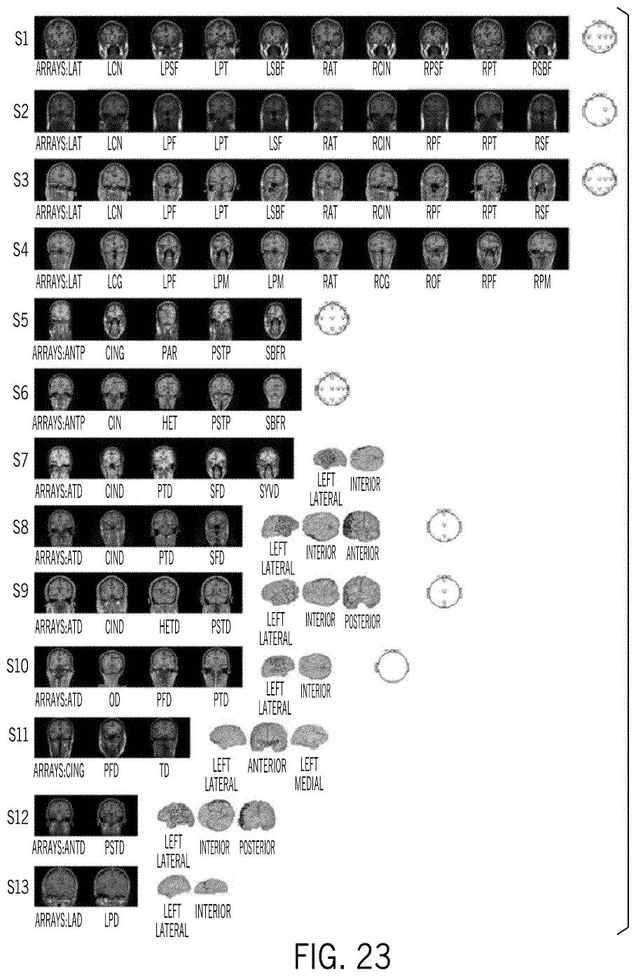

After LOC, novel alpha rhythms with two distinct phenomenologies are seen in this set of exemplars. An alpha band rhythm appears with a bursting pattern in all channels and with greatest strength in the cingulate cortex recording. The same pattern is seen in both the anterior and posterior surface EEG. A broadband rhythm in the hippocampus between slow frequencies (0.1-1 Hz) and high beta/low alpha frequencies (11-13 Hz) emerges after LOC. The phenomena in this subject's exemplars in temporal cortex, cingulate cortex, hippocampus, occipital cortex, and surface EEG are similar to those observed in all other subjects in the data set (FIG. 23).

The lower panel of FIG. 22 shows alpha power dynamics across LOC in all of the iEEG electrodes for this patient. Greatest alpha power prior to LOC (top row) occurs in occipital electrodes, and least power is in the cingulate cortex. The distribution is reversed after LOC. A similar distribution is seen in all the other subjects in the data set.

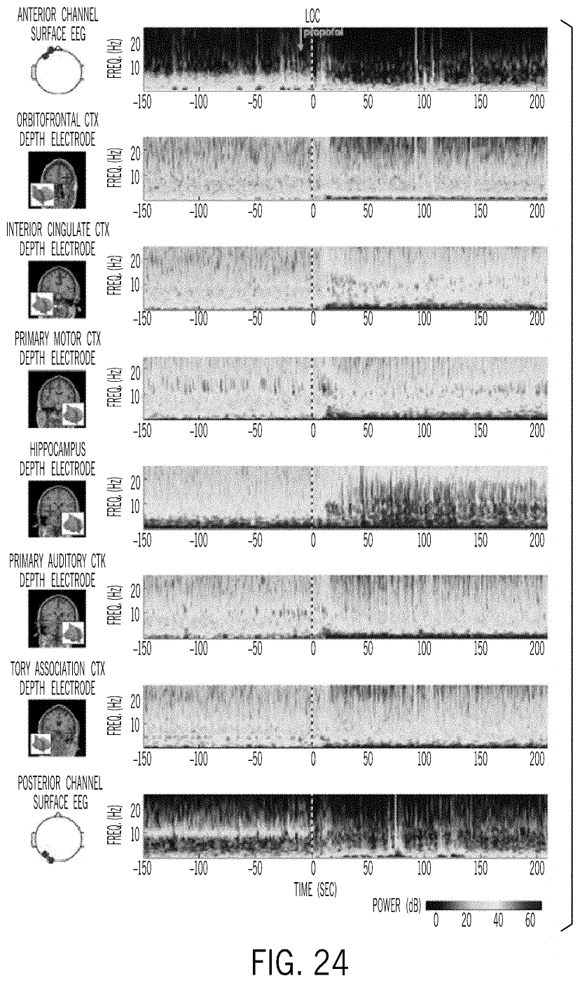

FIG. 24 shows spectrograms from exemplar recordings from patient 3, which demonstrate one feature not visible in the first patient's exemplars due to a different distribution of recording sites. In patient 3, a spectrogram from a motor cortex electrode shows activity consistent with the sensorimotor mu oscillation, which has been shown to occur at a higher frequency than occipital alpha and to be concurrent with beta power. In this recording, alpha power at a different frequency band occurs in the motor cortex after LOC, unlike in the auditory and visual cortex recordings in this and the previous patient. This result in motor and sensorimotor cortices is observed in numerous subjects, which may suggest a distinct behavior in sensorimotor mu producing sites and occipital alpha and temporal tau producing sites.

Anteriorization of EEG Occurs with Medialization and Anteriorization of iEEG

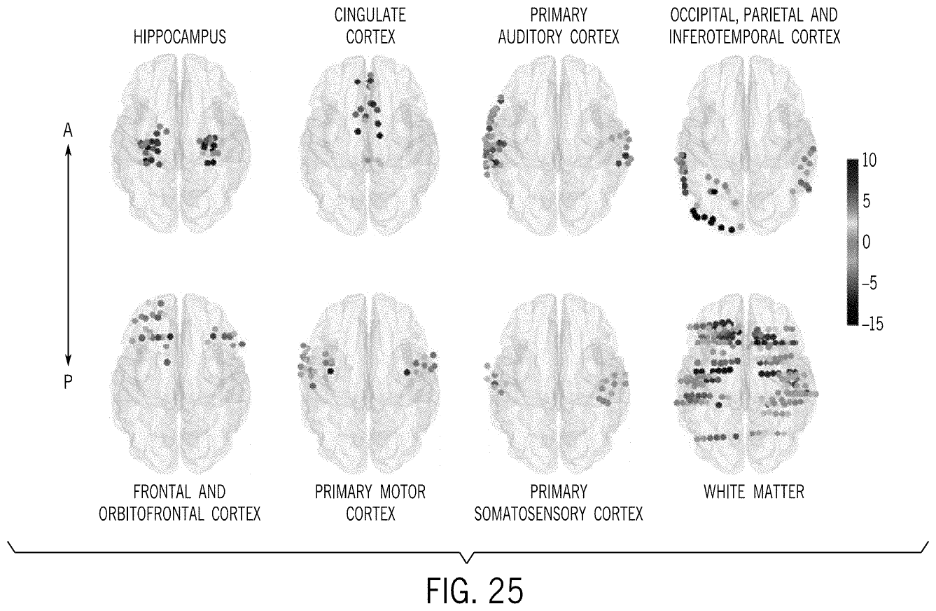

FIG. 25 shows a region-specific summary of the alpha power change during anesthesia. Median alpha power in the postinduction period is consistently increased in the hippocampus and cingulate cortex, as well as in medial white matter electrodes, by approximately 5 dB. Power is also consistently decreased in a broad region including occipital cortex, parietal cortex, and inferotemporal cortex, as well as in the primary auditory cortex. Activity in motor cortex, somatosensory cortex, and frontal gray matter increases in some channels and decreases in others. This pattern reflects a medialization of alpha power in the iEEG that is concurrent with the traditional anteriorization in EEG.

### A Novel Alpha Oscillation in Hippocampus During Propofol General Anesthesia

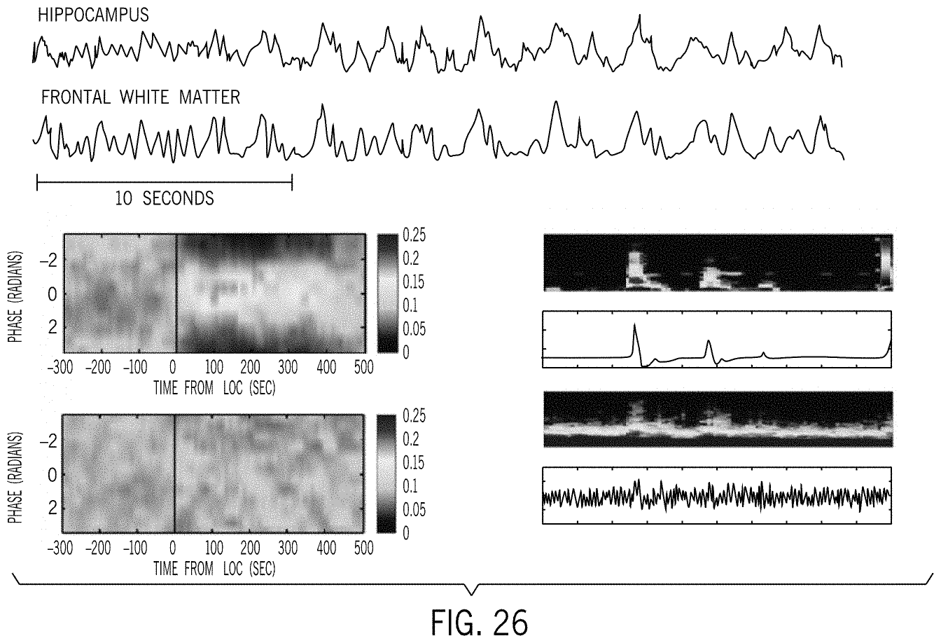

In all patients with electrodes in hippocampus a novel broadband rhythm including the alpha frequency was observed within +/−30 seconds around LOC. Like the fronto-medial alpha rhythms that occurred in the post-induction period, this novel hippocampal oscillation of propofol GA had an onset tightly linked with unconsciousness. However, the rhythm had several properties that distinguished it from the other alpha rhythms of GA and waking and these were consistent across all subjects. The hippocampal alpha rhythm was broadband, with nearly uniform power from slow (0.1-1) to alpha or low beta frequencies. This rhythm was less coupled to slow oscillation phase than in the signals recorded from locations in other cortical and subcortical regions (FIG. 26). The rhythm persisted during suppression periods of burst suppression.

Changes in Alpha Power are Tightly Linked with Timing of LOC

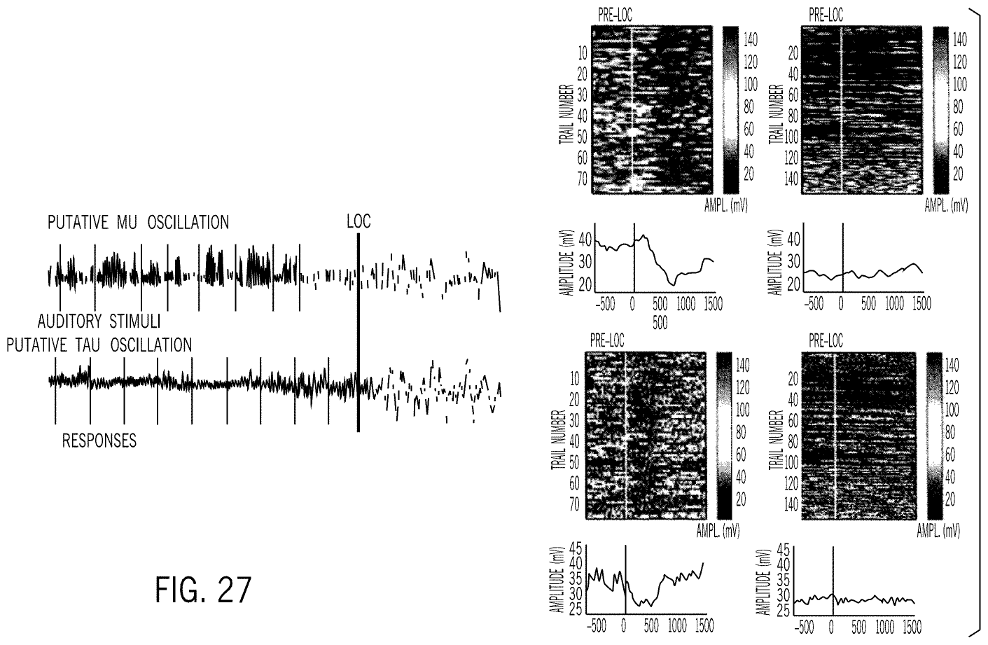

The exemplar figures show changes in an alpha oscillation that is tightly linked within +/−30 seconds to loss of responsiveness. In some patients (patients 1 and 3), the offset of an occipital alpha oscillation was most closely temporally linked with LOC and the onset of cingulate and hippocampal rhythms occurred after LOC while in others the pattern was reversed (patient 5). In all patients demonstrating mu and tau oscillations before induction of anesthesia, these oscillations were no longer significantly related to timing of stimuli after induction of anesthesia (FIG. 27).

### Discussion

We have examined human intracranial and EEG neural recordings during the transition to unconsciousness with propofol bolus, and demonstrated a) a disruption of the occipital, tau, and mu alpha rhythms of wakefulness within seconds surrounding LOC, and b) an emergence of de novo alpha frequency rhythms in the hippocampus and frontal midline structures including cingulate cortex, orbitofrontal and prefrontal cortex, and frontal white matter that are not observed in typical wakefulness. The distribution of changes in alpha power in the occipital lobe, auditory cortex, somatomotor cortex, hippocampus, and frontal midline regions were consistent across patients during the transition to unconsciousness in the clincial setting.

### Neurophysiological Mechanisms of Propofol Alpha Dynamics

We showed increased alpha power after LOC in anterior midline channels in cingulate cortex, frontal, prefrontal and orbitofrontal cortex, and frontal white matter channels. These regions receive projections from the mediodorsal nucleus of the thalamus, which may underlie a common alpha power dynamic driven via common thalamocortical projections. We observed region-specific differences between propofol's disruptive effects at LOC in the cortical regions that produce occipital alpha, tau, and mu oscillations in waking. In occipital alpha-and tau-producing cortical regions, alpha power was reduced after LOC, while in sensorimotor mu-producing cortical regions, in alpha power was for some patients and channels increased. Several neurophysiological mechanisms may underlie this distinction.

Occipital alpha-, tau-, and mu-producing regions of cortex receive projections from distinct thalamic relay nuclei, with the lateral geniculate nucleus projecting to the occipital cortex, the medial geniculate body projecting to auditory areas, and the ventral nuclei projecting to somatomotor areas. Furthermore, the waking alpha rhythms are thought to require connectivity between thalamic reticular neurons and thalamic relay neurons. For the mu rhythm in particular, inhibitory connectivity between reticular and relay nuclei is thought to play an important role in generating the bilaterlally incoherent, focally specific alpha rhythms during sensorimotor tasks. Finally, distinct effects of propofol on GABAergic cortico-cortico circuits may underlie propofol's distinct effects. In specific, cortico-cortical mechanisms are thought to be important in generating the slow cortical potential, which has been shown to gate higher frequency activity after propofol.

A Novel Hippocampal Alpha Rhythm with Propofol

We have reported a novel broadband rhythm localized to hippocampal channels that includes power in the alpha frequency band, emerges near LOC, persists through the suppression periods of burst suppression, and has slow oscillation coupling properties distinct from the fronto-medial alpha rhythm of propofol GA. Taken together, these features suggest that local hippocampal generators may play be implicated in this rhythm following LOC. Because in vitro experiments have not indicated the generation of alpha rhythms with the application of propofol to hippocampal slices while changes in higher frequency gamma oscillations have been shown, it can be hypothesized that an intact hippocampus is essential to generate the rhythm. An alternate hypothesis is that the effect has not been observed in vitro because of species-specific differences in hippocampal circuitry.

Some properties of this novel rhythm following propofol may be related to neural mechanisms that are functionally significant during waking. If the hypothesis of a local generator of the hippocampal rhythm is confirmed, the result would suggest that GABAergic hippocampal circuits have the capacity to generate oscillations in a broad frequency range, including alpha, and that hippocampal activity may simultaneously reflect locally generated rhythms and thalamically mediated alpha rhythms at distant cortical sites. Such a capacity may be mechanistically relevant to memory encoding and/or retrieval in wakefulness. A role of hippocampal alpha rhythms in memory processes has been previously reported though the effect is less well studied than the role of hippocampal theta in learning and memory. GABAergic circuits in the hippocampus have been implicated in several properties of hippocampal function in the waking state. The results presented here may inform future biophysical models of human hippocampal circuitry in response to GABA-ergic anesthetic agents in both anesthesia and waking.

The results of this study, in particular with respect to a novel anesthesia-related rhythm in hippocampus, need be interpreted with regards to the dataset of epileptic patients. What is known about GABA-ergic hippocampal networks is consistent with a specific effect in this region. To extend these results, subcortical source localization, animal studies, and epilepsy may affect results in several ways with respect to normal patients. Activity in the hippocampus may be distinct in power and frequency. We would hypothesize that the broadband, bilaterally coherent properties of this rhythm would be retained. We would also hypothesize that the top-edge frequency may be different, or that power may be decreased in normal controls.

Relationship with Sleep and Coma

The anterior medial pattern of alpha power that we have demonstrated after propofol LOC is not typically observed during sleep. Alpha rhythms that vary with sleep stage have a primarily occipital distribution unlike those we have shown in propofol general anesthesia. Certain variants of alpha-pattern coma have been described whose power distribution is similar to those we observe in this study. Postmortem neurohistology in alpha-pattern coma has suggested that widespread cortical, thalamic, and/or brainstem damage may underlie the fronto-medial power distribution, which is consistent with current theories of where propofol may act to disrupt GABA-ergic networks.

Relationship with Behavioral Changes of Anesthesia and Implications for Monitoring

The experimental protocol in this study allowed measurement of loss of responsiveness with a temporal resolution of several seconds. LOC was closely linked to increases in medio-frontal and hippocampal alpha power and decreases in occipital and tau alpha power. Specific disruption of occipital alpha and auditory tau, and changes in sensorimotor mu oscillations after LOC may be related separable behavioral components of anesthesia: loss of consciousness and akinesia. The disruption of task-related auditory tau oscillations and sensorimotor mu oscillations occurred abruptly within seconds of LOC. The disruptions of these rhythms may be related to the inability to perceive sensory stimuli and perform movements, and may link general anesthesia with disruptions in specific systems-level functional circuits. As such, the novel broadband rhythm observed in the hippocampus of all subjects may be related to anesthetic-induced amnesia. If the rhythm is related to propofol-induced amnesia, it may provide a novel target for anesthetic monitoring to prevent post-operative recall.

In summary, the present invention provides an electroencephalogram (EEG) marker, which may be related directly to the effect of an anesthetic drug, that can be computed in real-time and used to generate an error signal. This error signal or the difference between the measured marker value and the marker value targeted can be used to maintain the desired anesthetic state. In one aspect, the error signal is processed in real-time to adjust the rate of drug administration from a computer-controlled infusion device. If the pharmacokinetics of the underlying system and the response of the closed-loop controller are well characterized, it is possible to control the EEG marker and maintain the brain in the specified anesthetic state for as long as desired.

In accordance with one aspect of the present invention, a new metric, for example, burst suppression probability (BSP), may be utilized in new and different methodologies not contemplated in traditional systems relying on old metrics, such as burst suppression ratio (BSR). In addition, a system and method is provided for converting EEG bursts and suppressions into binary time series in real-time. The binary time series serves as an input to a burst suppression probability (BSP) algorithm. The BSP algorithm provides a second-to-second estimate of the brain's state of burst suppression using a state space model for binary and point process observations. An analysis paradigm is provided to estimate model parameters using an expectation maximization (EM) algorithm. The EM algorithm provides confidence intervals to facilitate formal statistical comparisons between the BSP estimated at any two time points.

Additionally, the present invention provides a closed-loop system for monitoring and controlling administration of anesthetic compounds and/or a current or desired state of a patient receiving the anesthetic compounds. The closed-loop system of the present invention can be designed to utilize burst suppression and the BSP as control mechanisms. Multi-compartment, burst-suppression-specific models for various drugs have been implanted using pharmacokinetic population models. More particularly, drug-specific proportional-integral (PI) controllers have been designed to maintain a specific, BSP target level. Within this control system, an error signal can be reliably computed using a binary filter algorithm of the present invention to estimate the BSP from EEG recordings. Additionally, the present invention provides a system and method for determining the state of a patient's brain under anesthesia using readily-available monitoring information, such as from a patient's electroencephalography (EEG).

The present invention also provides a two-compartment model composed of a central plasma compartment and an auxiliary effect-site compartment connected by a first-order transfer process and may ignore any other peripheral drug distribution compartments for simplicity and effectiveness, as desired. The model can be used in a closed-loop system to advantageously utilize a binary filter algorithm to convert EEG signals in real-time into an estimate of the BSP. Again, the greater the BSP the greater the level of suppression, and hence the BSP provides a specific quantitative measure of the depth of the induced medical coma. A mathematical model that parametrically relates anesthetic infusion dynamics to the burst suppression probability is provided. Signal processing algorithms for extracting burst suppression morphology from the EEG are also provided. The controller can be designed to regulate the BSP in real-time using feedback control based on the binary filter output.

The present invention recognizes that anesthetic compounds induce different signatures in physiological characteristics of the patient under anesthesia and aids interpretation of such information. For example, the present invention is adaptable for use with a diverse list of clinical applications including, for example, control of medical coma, control of intensive care unit (ICU) sedation, control of general anesthesia in the operating room (OR), control of sedation for outpatient procedures, and the like. Unlike previous attempts to control depth of anesthesia and other CLAD systems, this invention is envisioned for use in conjunction with the medical judgment of a clinician, by providing an “autopilot” to help maintain specific physiologically-defined brain states selected by the clinician. This CLAD system thereby enhances the ability of the clinician to manage the care of the patient. Using the physiological characteristics and signatures associated with the selected anesthetic compound, the present invention aids the correlation of the physiological characteristics and signatures to a state of the patient's brain. Further still, the present invention provides systems and method for actively controlling and/or inducing an active arousal in a patient under the influence of anesthetic compounds.

In accordance with another aspect of the present invention, an automated system is provided for the maintenance of burst suppression to achieve a precise or desired patient state. The present invention can be used to identify and track burst suppression with respect to particular in disease states, such as hypothermia, general anesthesia, medical coma, and the like. As such, the present invention can be used, for example, to implement a rational approach to the use of hypothermia for neuro-protective purposes during cardiopulmonary bypass, total circulatory arrest and treatment of post-anoxic encephalopathy.

More specifically, the above-described closed-loop system can advantageously utilize the binary filter algorithm described above to convert the EEG in real-time into an estimate of the BSP. Again, the greater the BSP the greater the level of suppression, and hence the BSP provides a specific quantitative measure of the depth of the induced medical coma. A mathematical model that parametrically relates anesthetic infusion dynamics to the burst suppression probability is provided. Signal processing algorithms for extracting burst suppression morphology from the EEG are also provided. The controller can be designed to regulate the BSP in real-time using feedback control based on the binary filter output.

In addition to the above-described capabilities, the closed-loop system can utilize an extended algorithm for a complete multi-drug solution and active awaking. Specifically, the present invention recognizes that anesthetic compounds induce different signatures in physiological characteristics of the patient under anesthesia and aids interpretation of physiological characteristics and signatures therein based on a selected anesthesia compound. Using the physiological characteristics and signatures associated with the selected anesthesia compound, the present invention aids in relating of the physiological characteristics and signatures to a state of the patient's brain, such as described in detail in co-pending application PCT/US12/36854, entitled, “SYSTEM AND METHOD FOR TRACKING BRAIN STATES DURING ADMINISTRATION OF ANESTHESIA” and incorporated herein by reference in its entirety.

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A system for monitoring a patient experiencing an administration of at least one drug having anesthetic properties, the system comprising:
a plurality of sensors configured to acquire at least electroencephalogram (EEG) signals from the patient;
a user interface configured to receive an indication of at least one characteristic of the patient and at least one drug administered to the patient;
a processor configured to:
receive data corresponding to the EEG signals and the indication;
assemble, using the data received, one or more sets of EEG time-series;
select alpha frequency signals from the one or more sets of EEG time-series;
analyze the alpha frequency signals to determine signatures particular to the at least one drug administered;
based on the signatures and the indication, identify at least one of a current state and a predicted future state of the patient induced by the at least one drug; and
generate a report indicating at least one of the current state and the predicted future state of the patient.

2. The system of claim 1, wherein the processor is further configured to use the current state, determined signatures, and the indication in a model to identify the predicted future state of the patient.

3. The system of claim 2, wherein the processor is further configured to determine a burst suppression probability using the model and the one or more sets of EEG time-series.

4. The system of claim 1, wherein the processor is further configured to transform the one or more sets of EEG time-series into a spectrogram and analyze the spectrogram to identify at least one of the current state and the predicted future state of the patient.

5. The system of claim 1, wherein the processor is further configured to perform a phase-amplitude analysis on the one or more sets of EEG time-series to measure a phase-amplitude coupling in a time-resolved fashion to identify modes of phase-amplitude coupling corresponding to at least one of the current state and the predicted future state of the patient.

6. The system of claim 1, wherein the processor is further configured to determine coherence information with respect to the one or more sets of EEG time-series and analyze the coherence information using the determined signatures to determine at least one of the current state and the predicted futures state of the patient.

7. The system of claim 1, wherein indication of at least one of a characteristic of the patient and the at least one drug having anesthetic properties includes at least one of an age of the patient, a drug selecting from the list consisting essentially of Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and drug administration information including at least one of drug timing, drug dose, and drug administration rate.

8. The system of claim 1, wherein the processor is further configured to generate commands for a drug delivery system, based on the report, to direct the administration of the least one drug by the drug delivery system to the patient to attain the predicted future state.

9. The system of claim 1, wherein the processor is configured to implement a dynamic processing method to characterize the patient as exhibiting a predetermined behavioral dynamic, and wherein the behavioral dynamic includes at least one of a loss consciousness and recovery of consciousness.

10. The system of claim 1, wherein the processor is further configured to generate a report that indicates spatiotemporal activity at different states of the patient receiving the drug.

11. A method for monitoring a patient experiencing an administration of at least one drug having anesthetic properties using a monitoring system, the method comprising:
receiving data corresponding to EEG signals acquired from a patient and an indication of at least one characteristic of the patient and the at least one drug having anesthetic properties;
assembling, using the data received, one or more sets of EEG time-series;
selecting alpha frequency signals from the one or more sets of EEG time-series;
analyzing the alpha frequency signals to determine signatures particular to the at least one drug administered;
based on the signatures and the indication, identifying at least one of a current state and a predicted future state of the patient induced by the at least one drug; and
generating a report indicative of the at least one of the current state and the predicted future state of the patient.

12. The method of claim 11, wherein the method further comprises utilizing the current state, determined signatures, and the indication in a model to identify the predicted future state of the patient.

13. The system of claim 12, wherein the method further comprises determining a burst suppression probability using the model and the one or more sets of EEG time-series.

14. The method of claim 11, wherein the method further comprises transforming the one or more sets of EEG time-series into a spectrogram and analyzing the spectrogram to determine at least one of the current state and the predicted future state of the patient.

15. The method of claim 11, wherein the method further comprises performing a phase-amplitude analysis on the one or more sets of EEG time-series to measure a phase-amplitude coupling in a time-resolved fashion to identify modes of phase-amplitude coupling corresponding to at least one of the current state and the predicted future state of the patient.

16. The method of claim 11, wherein the method further comprises determining coherence information with respect to the one or more sets of EEG time-series, and analyzing the coherence information using the signatures to determine at least one of the current state and the predicted futures state of the patient.

17. The method of claim 11 wherein indication of at least one of a characteristic of the patient and the at least one drug having anesthetic properties includes at least one of an age of the patient, a drug selecting from the list consisting essentially of Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and drug administration information including at least one of drug timing, drug dose, and drug administration rate.

18. The method of claim 11, wherein the method further comprises generating commands for a drug delivery system, based on the report, to direct the administration of the least one drug by the drug delivery system to the patient to attain the predicted future state.

19. A system for monitoring and controlling a patient experiencing an administration of at least one drug having anesthetic properties, the system comprising:
a plurality of sensors configured to at least acquire electroencephalogram (EEG) signals from the patient;
a user interface configured to receive an indication of at least one characteristic of the patient and at least one drug administered to the patient;
at least one processor configured to:
receive data corresponding to the EEG signals and the indication;
assemble, using the data received, one or more sets of EEG time-series;
select alpha frequency signals from the one or more sets of EEG time-series;
analyze the alpha frequency signals to determine signatures particular to the at least one drug administered;
based on the signatures and the indication, identify a current state of the patient induced by the at least one drug; and
control the administration of the least one drug to attain a predicted future state for the patient; and
generate a report indicating at least one of the current state and the predicted future state of the patient induced by the at least one drug.

20. The system of claim 19, wherein the at least one processor is further configured to utilize the current state, the signatures, and the indication in a model to identify the predicted future state.
